(19) United States
(12) Patent Application Publication
(10) Pub. No.: US 2023/0104813 A1
(43) Pub. Date: Apr. 6, 2023

(54) OXYGEN CONCENTRATOR WITH A USER-REPLACEABLE DESICCANT RECEPTACLE

(71) Applicant: ResMed Asia Pte. Ltd., Singapore (SG)

(72) Inventors: Devona Yinqiu Poon, Singapore (SG); Rex Dael Navarro, Singapore (SG); Wai Loon Ooi, Singapore (SG); Shayan Miaralipour, Bella Vista NSW (AU)

(21) Appl. No.: 17/907,165

(22) PCT Filed: Mar. 24, 2021
(86) PCT No.: PCT/IB2021/052456

§ 371 Date: Sep. 23, 2022

Related U.S. Application Data
(60) Provisional application No. 63/000,598, filed on Mar. 27, 2020.

(51) Int. Cl. A61M 16/10; B01D 53/04; B01D 53/053; B01D 53/26; A61M 16/20

(52) U.S. Cl. CPC A61M 16/101; B01D 53/0415; B01D 53/053; B01D 53/261; A61M 16/202; A61M 2016/0033

(57) ABSTRACT

A user-replaceable receptacle for an oxygen concentrator includes a containment structure and a desiccant disposed within the containment structure. An inlet end of the containment structure allows feed gas to be introduced into the desiccant. An outlet end of the containment structure allows the feed gas to exit the containment structure. A connection mechanism couples the outlet end of the containment structure to a gas separation adsorbent. The connection mechanism is operable between an unconnected position and a connection position. The desiccant in the user-replaceable receptacle removes water moisture from the feed gas prior to exiting the outlet end of the containment structure, thereby reducing exposure of the gas separation adsorbent to water.

### CROSS-REFERENCE TO RELATED APPLICATIONS

This application claims priority to, and benefit of, U.S. Provisional Patent Application No. 63/000,598, filed Mar. 27, 2020, which is hereby incorporated by reference herein in its entirety.

### FIELD OF THE TECHNOLOGY

The present technology relates generally to methods and apparatus for treating respiratory disorders, such as those involving gas adsorption or controlled pressure and/or vacuum swing adsorption. Such methodologies may be implemented in an oxygen concentrator using one or more sieve beds. In some examples, the technology more specifically concerns such methods and apparatus for a portable oxygen concentrator or a sieve bed assembly including a user-replaceable desiccant receptacle.

### BACKGROUND

### Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include asthma, respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Asthma is a common long-term inflammatory disease of the airways of the lungs. It is characterized by variable and recurring symptoms, reversible airflow obstruction, and easily triggered bronchospasms. Symptoms include episodes of wheezing, coughing, chest tightness, and shortness of breath.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterized by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterized by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterized by muscle impairment that worsens over years and only mildly reduces life expectancy (e.g. Limb girdle, Facioscapulohumeral and Myotonic muscular dystrophy). Symptoms of respiratory failure in NMD include: increasing generalized weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterized by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Therapies

Various respiratory therapies, such as Non-invasive ventilation (NIV), Invasive ventilation (IV), and High Flow Therapy (HFT) have been used to treat one or more of the above respiratory disorders. These therapies may be provided with supplemental oxygen. Alternatively, or in conjunction therewith, i.e. as a concomitant therapy, oxygen therapy may be administered in its own right, either via a stationary i.e. (non-portable) oxygen concentrator or a portable oxygen concentrator.

### Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous or pulsed flow of oxygen enriched air at a specified oxygen concentration (from 21%, the oxygen fraction in ambient air, to 100%) at a specified flow rate (e.g., 1 litre per minute (LPM), 2 LPM, 3 LPM, etc., either as a continuous flow or pulsed flow equivalent) to be delivered to the patient's airway. With on-demand or pulsed flow, delivery may be measured by the size (in milliliters) of the “bolus” of oxygen per breath. Oxygen delivery may be achieved by a stationary oxygen concentrator or a portable oxygen concentrator

### Supplementary Oxygen

For certain patients, oxygen therapy may be combined with a respiratory pressure therapy or HFT by adding supplementary oxygen to the pressurized flow of air. When oxygen is added to respiratory pressure therapy, this is referred to as RPT with supplementary oxygen. When oxygen is added to HFT, the resulting therapy is referred to as HFT with supplementary oxygen.

### Respiratory Therapy Systems

These respiratory therapies may be provided by a respiratory therapy system or device. Such systems and devices may also be used to screen, diagnose, or monitor a condition without treating it.

A respiratory therapy system as described herein may comprise an oxygen source, an air circuit, and a patient interface.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds (22.68 kg) when mounted.

Oxygen concentrators have been in use for about 50 years to supply oxygen for respiratory therapy. Oxygen concentrators may implement processes such as vacuum swing adsorption (VSA), pressure swing adsorption (PSA), or vacuum pressure swing adsorption (VPSA). For example, pressure swing adsorption may involve using one or more compressors to increase gas pressure inside one or more canisters that contain(s) particles of a gas separation adsorbent (i.e., a sieve bed). As the pressure increases, certain molecules in the gas may become adsorbed onto the gas separation adsorbent. Removal of a portion of the gas in such a canister under the pressurized conditions allows separation of the non-adsorbed molecules from the adsorbed molecules. The gas separation adsorbent may be regenerated by reducing the pressure, which reverses the adsorption (i.e., desorption) of molecules from the adsorbent. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for purging of the beds.

Ambient air, an ambient gas (AG), usually includes approximately 78% nitrogen and 21% oxygen with the balance comprised of argon, carbon dioxide, water vapor (i.e. water moisture) and other trace gases. If a gas mixture such as air, for example, is passed under pressure through a vessel containing a gas separation adsorbent bed that attracts nitrogen more strongly than it does oxygen, part or all of the nitrogen will stay in the bed, and the gas coming out of the vessel, which may be product gas (PG), will be enriched in oxygen. When the bed reaches the end of its capacity to adsorb nitrogen, it can be regenerated by reducing the pressure, thereby releasing the adsorbed nitrogen, which may be vented from the system, such as to atmosphere, as a waste gas (WG). It is then ready for another cycle of producing oxygen enriched gas. By alternating canisters in a two-canister system, one canister can be collecting oxygen while the other canister is being purged (resulting in a continuous separation of the oxygen from the nitrogen). In this manner, oxygen can be accumulated, such as in a storage container or other pressurizable vessel or conduit coupled to the canisters, from the ambient air for a variety of uses include providing supplemental oxygen to users.

Traditional oxygen concentrators have been bulky and heavy, making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and can provide mobility for patient users. In order to make these devices small for mobility, the various systems necessary for the production of oxygen enriched air are condensed. POCs seek to utilize their produced oxygen as efficiently as possible, in order to minimize weight, size, and power consumption. In some cases, this may be achieved by delivering the oxygen as series of pulses or “boli”, each bolus timed to coincide with the start of inspiration. This therapy mode is known as pulsed or demand (oxygen) delivery (POD), in contrast with traditional continuous flow delivery more suited to stationary oxygen concentrators.

### Air Circuit

An air circuit is a conduit or a tube constructed and arranged to allow, in use, a flow of air to travel between two components of a respiratory therapy system such as the oxygen source and the patient interface. In some cases, there may be separate limbs of the air circuit for inhalation and exhalation. In other cases, a single limb air circuit is used for both inhalation and exhalation.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### SUMMARY

According to one aspect of the present technology, a sieve bed assembly for a portable oxygen concentrator includes at least one canister. Each canister of the sieve bed assembly comprises an inlet, an outlet, and a housing defining an internal chamber between the inlet and the outlet. The internal chamber comprises a first section disposed adjacent to the inlet. The first section includes a user-replaceable receptacle containing a desiccant. The internal chamber further comprises a second section disposed adjacent to the outlet. The second section includes a gas separation adsorbent. The inlet and the outlet are in fluid communication with the internal chamber. The user-replaceable receptacle is disposed between the inlet and the gas separation adsorbent to remove water from fluid entering the internal chamber via the inlet.

According to another aspect of the present technology, a portable oxygen concentrator comprises a compression system including a compressor, wherein the compressor is coupled to a sieve bed assembly. The sieve bed assembly includes at least one canister, each canister of the sieve bed assembly comprising an inlet, an outlet, and a housing defining an internal chamber between the inlet and the outlet. The internal chamber comprises a first section disposed adjacent to the inlet. The first section includes a user-replaceable receptacle containing a desiccant. The internal chamber further comprises a second section disposed adjacent to the outlet. The second section includes a gas separation adsorbent. The inlet and the outlet are in fluid communication with the internal chamber. The user-replaceable receptacle is disposed between the inlet and the gas separation adsorbent to remove water from fluid entering the internal chamber via the inlet. The gas separation adsorbent may include a gas separation adsorbent cartridge, and the gas separation adsorbent may include a crystalline substance with pores that adsorb gaseous molecules larger than oxygen gas.

According to yet another aspect of the present technology, a user-replaceable receptacle for a portable oxygen concentrator comprises a containment structure comprising an inlet and an outlet, and a desiccant disposed within the containment structure. An inlet of the containment structure allows feed gas to be introduced into the desiccant. An outlet of the containment structure allows the feed gas to exit the containment structure. The user-replaceable receptacle further comprises a connection mechanism for coupling the outlet of the containment structure to a gas separation adsorbent. The connection mechanism is operable between an unconnected position and a connected position such that when the connection mechanism is in the connected position, water entering the gas separation adsorbent is reduced. The desiccant in the user-replaceable receptacle removes water from the feed gas, thereby reducing exposure of the gas separation adsorbent to water.

The above summary is not intended to represent every aspect of the present technology. Rather, the foregoing summary merely provides an example of some of the novel aspects and features set forth herein. The above features and advantages, and other features and advantages of the present technology, will be readily apparent from the following detailed description of representative embodiments and modes for carrying out the present invention, when taken in connection with the accompanying drawings and the appended claims.

### BRIEF DESCRIPTION OF THE DRAWINGS

Advantages of the present technology will become apparent to those skilled in the art with the benefit of the following detailed description of implementations and upon reference to the accompanying drawings in which similar reference numerals indicate similar components:

FIG. 1A depicts an oxygen concentrator in accordance with one form of the present technology.

FIG. 1B is a schematic diagram of the components of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 1I is a perspective view of a disassembled canister system for the oxygen concentrator of FIG. 1A.

FIG. 1J is an end view of the canister system of FIG. 1I.

FIG. 1K is an assembled view of the canister system end depicted in FIG. 1J.

FIG. 1L is a view of an opposing end of the canister system of FIG. 1I to that depicted in FIGS. 1J and 1K.

FIG. 1M is an assembled view of the canister system end depicted in FIG. 1L.

FIG. 1N depicts an example control panel for the oxygen concentrator of FIG. 1A.

FIG. 2 depicts a connected POC therapy system that includes the oxygen concentrator of FIG. 1A.

FIG. 3A depicts an oxygen concentrator in accordance with another exemplary form of the present technology.

FIG. 3B depicts an exploded view of the main components of the oxygen concentrator of FIG. 3A.

FIG. 3C is a perspective view of a sieve bed assembly for the oxygen concentrator of FIG. 3A.

FIG. 3D is an exploded view of the main components of the sieve bed assembly of FIG. 3C.

FIG. 3E is a perspective view of a cross-section of the sieve bed assembly of FIG. 3C.

FIG. 4A depicts an oxygen concentrator in accordance with yet another exemplary form of the present technology.

FIG. 4B depicts is an exploded view of the main components of the oxygen concentrator of FIG. 4A.

FIG. 4C is a perspective view of a sieve bed assembly for the oxygen concentrator of FIG. 4A.

FIG. 4D is a perspective view of a cross-section of the sieve bed assembly of FIG. 4C.

FIG. 5 depicts a perspective cross-sectional view of the exemplary sieve bed assembly in FIGS. 3C-3E including a user-replaceable desiccant receptacle.

FIG. 6 depicts a perspective cross-sectional view of the exemplary sieve bed assembly in FIGS. 4C-4D including a user-replaceable desiccant receptacle.

FIG. 7 is a schematic diagram of select exemplary inlet components for introducing fluid (e.g. feed gas) into an exemplary sieve bed assembly.

FIGS. 8 to 11 depict exemplary locking and/or sealing mechanisms between a user-replaceable desiccant receptacle and a gas separation adsorbent.

FIGS. 12A and 12B depict an exemplary swinging door for a sieve bed assembly to allow an end-user to replace a desiccant receptacle.

FIGS. 13A and 13B depict an exemplary sieve bed assembly with a rotatable top cap to allow an end-user to gain access to the internal chamber to replace a desiccant receptacle.

FIGS. 14A and 14B depict an exemplary sieve bed assembly with a removable cap to allow an end-user to gain access to the internal chamber to replace a desiccant receptacle.

FIGS. 15A and 15B depict another exemplary sieve bed assembly with removable cap to allow an end-user to gain access to the internal chamber to replace a desiccant receptacle.

While the technology may be implemented with various modifications and alternative forms, specific aspects thereof are shown by way of example in the drawings and are described in detail herein. It should be understood, however, that the drawings and detailed description thereto are not intended to limit the technology to the particular form disclosed. Various modifications, equivalents, and alternatives may be implemented by combining any of the disclosed features of any of the specific examples described.

### DETAILED DESCRIPTION

An example adsorption device of the present technology involving an oxygen concentrator may be considered in relation to the examples of the figures. The examples of the present technology may be implemented with any of the following structures and operations.

### Outer Housing

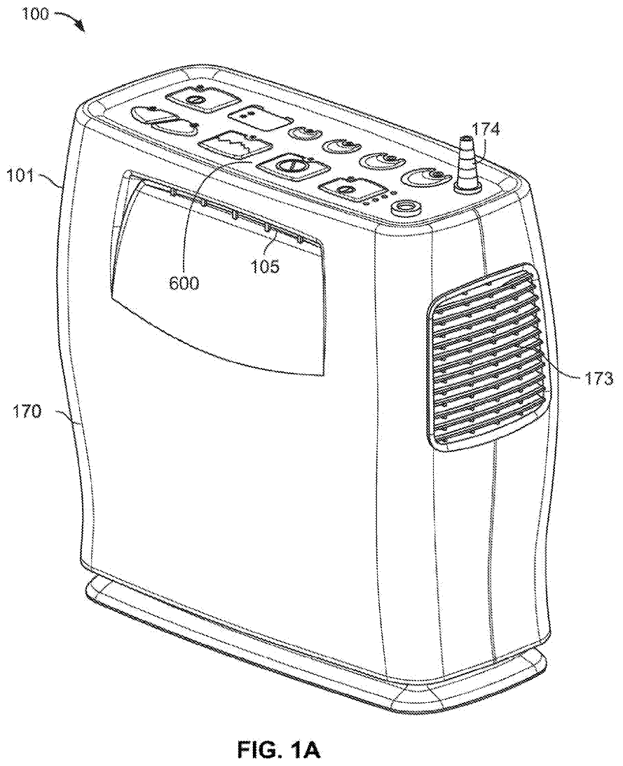

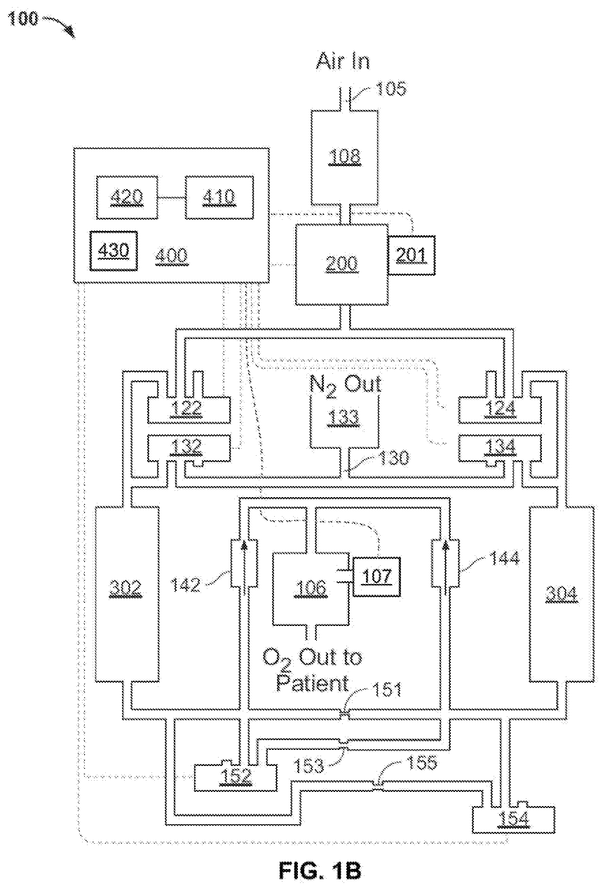

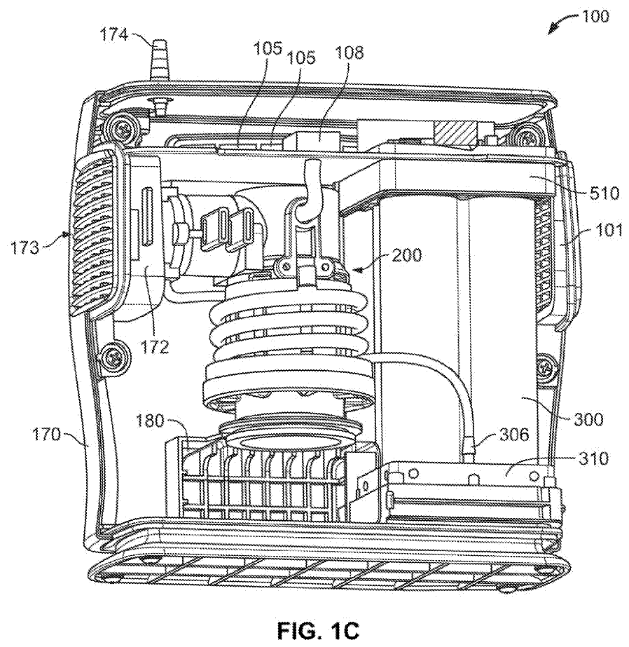

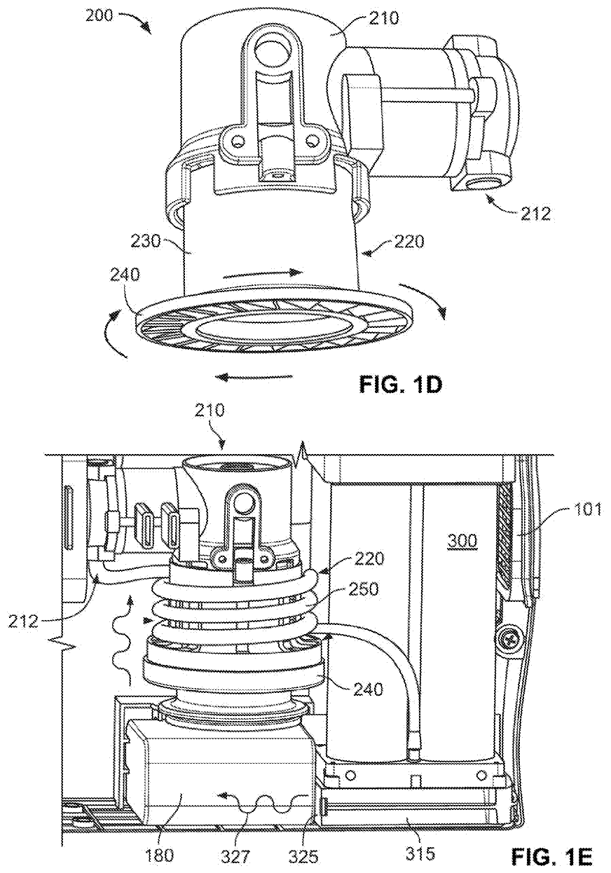

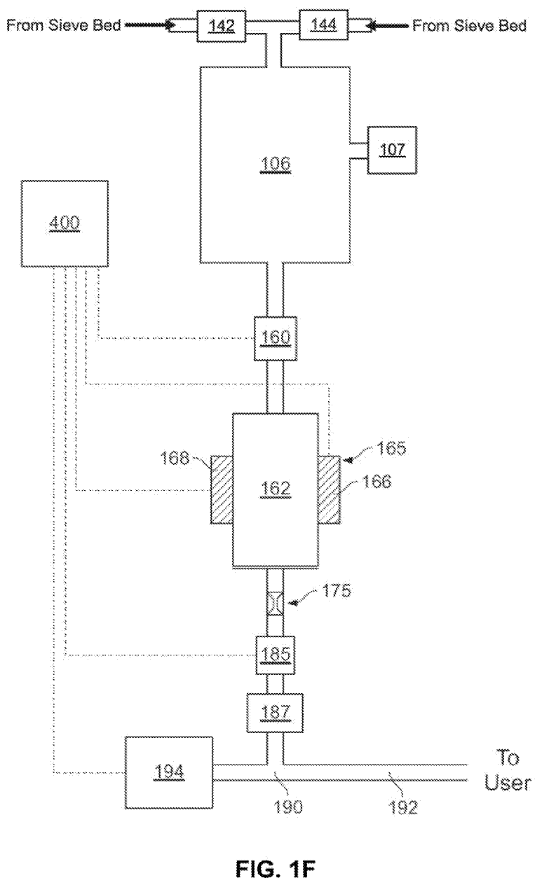

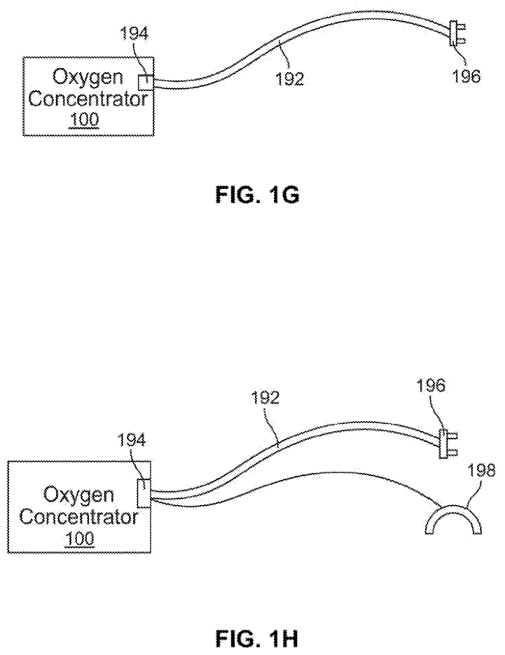

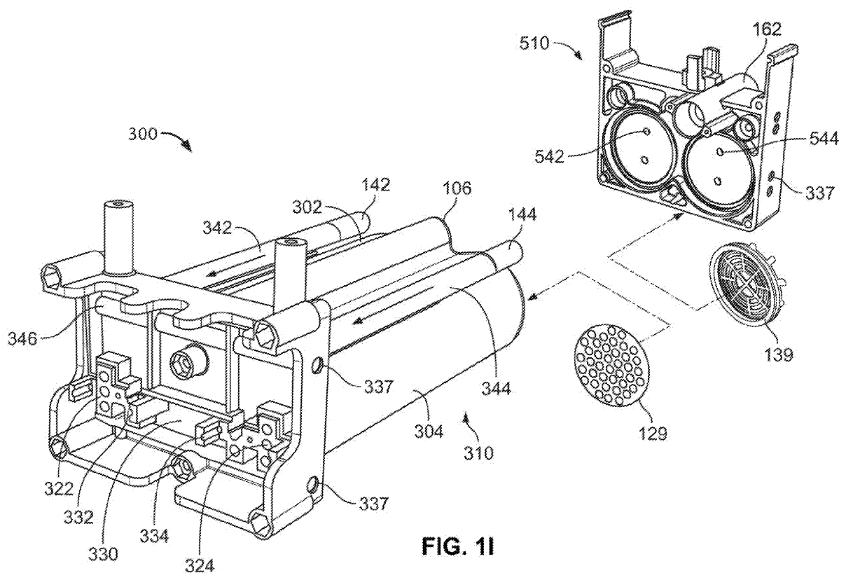

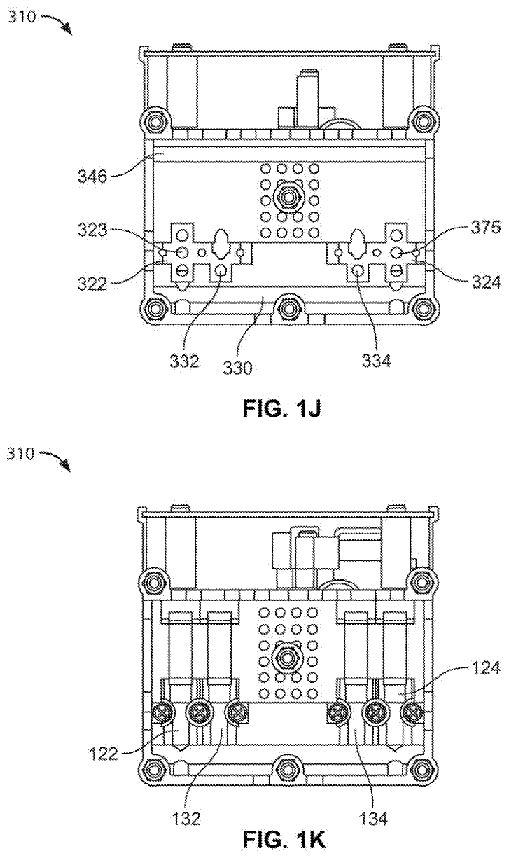

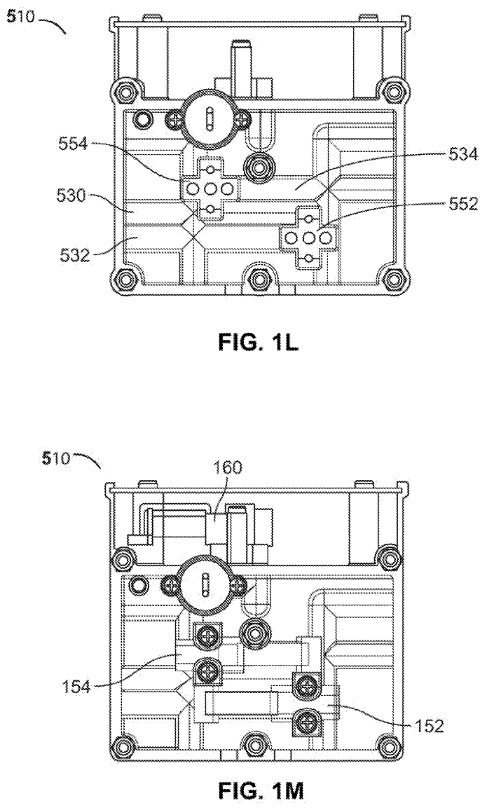

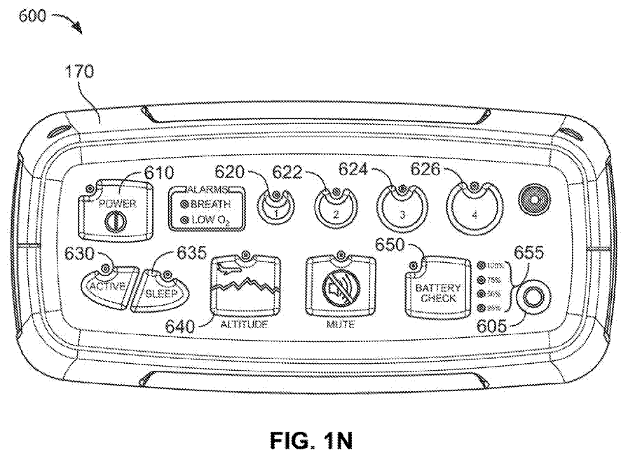

FIG. 1A depicts an implementation of an outer housing 170 of an oxygen concentrator 100. In some implementations, outer housing 170 may be comprised of a light-weight plastic. Outer housing 170 includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Components

FIG. 1B illustrates a schematic diagram of components of an oxygen concentrator 100, according to an implementation. In some implementations, such a device may be implemented with or without pneumatically piloted control valves. Oxygen concentrator 100 may concentrate oxygen within an air stream to provide oxygen enriched air to a user. As used herein, “oxygen enriched air” is a gas mixture composed of at least about 50% oxygen, at least about 60% oxygen, at least about 70% oxygen, at least about 80% oxygen, at least about 87% oxygen, at least about 90% oxygen, at least about 95% oxygen, at least about 98% oxygen, or at least about 99% oxygen.

Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds (9.07 kg), less than about 15 pounds (6.80 kg), less than about 10 pounds (4.54 kg), or less than about 5 pounds (2.27 kg). In an implementation, oxygen concentrator 100 has a volume of less than about 1000 cubic inches (0.0164 cubic meters), less than about 750 cubic inches (0.0123 cubic meters); less than about 500 cubic inches (0.0082 cubic meters), less than about 250 cubic inches (0.0041 cubic meters), or less than about 200 cubic inches (0.0033 cubic meters).

Oxygen enriched air, which may be considered a product gas (PG), may be produced from ambient air by pressurizing ambient air in canisters 302 and 304, which include a gas separation adsorbent. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a moisture adsorbent material (such as a polymer water adsorbent material or a zeolite material) may be used to both adsorb moisture, i.e. water, from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch (psi) (89.6-137.9 kPa). Other pressures may also be used, depending on the type of gas separation adsorbent disposed in the canisters.

Coupled to each canister 302/304 are inlet valves 122/124 and outlet valves 132/134. As shown in FIG. 1B, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Plunger valves offer advantages over other kinds of valves by being quiet and having low slippage. Other types of valves, however, may be used such as one or more valves that have pneumatic piloted controls.

In some implementations, such as in relation to the generation of electrical valve activation signals for electro-mechanical valves, a two-step valve actuation voltage may be generated to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the battery. When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for generating control signals for operating inlet valves 122 and 124 out of phase with each other, i.e., when one of inlet valves 122 or 124 is opened, the other valve is closed such as when electro-mechanical valve(s) are used. During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400. The controller 400 may include a transceiver 430 that may communicate with external devices to transmit data collected by the processor 410 or receive instructions from an external device for the processor 410.

Check valves 142 and 144 are coupled to canisters 302 and 304, respectively. Check valves 142 and 144 are one-way valves that are passively operated by the pressure differentials that occur as the canisters are pressurized and vented. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits or prevents back flow of the fluid. The term “fluid” may include a gas or a mixture of gases (such as, air). Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi (6.9 kPa). The break pressure in the reverse direction is greater than 100 psi (689.5 kPa). It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302, outlet valve 132 is opened allowing pressurized gas (mainly nitrogen) to exit the canister to atmosphere through concentrator outlet 130. In an implementation, the vented gases may be directed through muffler 133 to reduce the noise produced by releasing the pressurized gas from the canister. As gas is released from canister 302, the pressure in the canister 302 drops, allowing the nitrogen to become desorbed from the gas separation adsorbent. The released nitrogen exits the canister through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, a canister may be further purged of nitrogen using an oxygen enriched air stream that is introduced into the canister from the other canister.

In an exemplary implementation, a portion of the oxygen enriched air may be transferred from canister 302 to canister 304 when canister 304 is being vented of nitrogen. Transfer of oxygen enriched air from canister 302 to 304 during venting of canister 304, helps to further purge nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ (0.022 cm) which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated). In general, a flow restrictor allows flow in either direction of a pneumatic path that it restricts but its reduced size relative to the pneumatic path on either side of the restrictor provides a resistance to the continuous flow through it.

Flow of oxygen enriched air between the canisters may also be controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalizing the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may condense inside the canister as the air cools. Condensation of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurizing both canisters prior to shut down. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shut down, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. Oxygen concentrator 100 includes a compression system 200, a canister assembly 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove moisture, i.e. water, from the incoming air. Oxygen concentrator 100 may further include fan 172 used to vent air and other gases from the oxygen concentrator via outlet 173.

### Compression System

In some implementations, compression system 200 includes one or more compressors. In another implementation, compression system 200 includes a single compressor, coupled to all of the canisters of canister system 300. Turning to FIGS. 1D and 1E, a compression system 200 is depicted that includes compressor 210 and motor 220. Motor 220 is coupled to compressor 210 and provides an operating force to the compressor to operate the compression mechanism. For example, motor 220 may be a motor providing a rotating (or rotatable) component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 1B, which sends operating signals to the motor to control the operation of the motor. For example, controller 400 may send signals to motor 220 to: turn the motor on, turn motor the off, and set the operating speed of motor. Thus, as illustrated in FIG. 1B, the compression system 200 may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system 200 via the motor 220 based on the speed signal and/or any other sensor signal of the oxygen concentrator, such as a pressure sensor (e.g., accumulator pressure sensor 107). Thus, as illustrated in FIG. 1B, the controller 400 receives sensor signals, such as a speed signal from the speed sensor 201 and accumulator pressure signal from the accumulator pressure sensor 107. With such signal(s), the controller 400 may implement one or more control loops (e.g., feedback control) for operation of the compression system 200 based on sensor signals such as accumulator pressure and/or motor speed as described in more detail herein.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as a power source for oxygen concentrators due to their long life and light weight. Lithium ion battery packs, however, are dangerous at elevated temperatures and safety controls are employed in oxygen concentrator 100 to shut down the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply and thus shorten the portable usage time of the oxygen concentrator. In an implementation, a passive cooling system may be used that takes advantage of the mechanical power produced by motor 220. Referring to FIGS. 1D and 1E, compression system 200 includes motor 220 having an external rotatable armature 230. Specifically, armature 230 of motor 220 (e.g. a DC motor) is wrapped around the stationary field that is driving the armature. Since motor 220 is a large contributor of heat to the overall system it is helpful to pull heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotatable armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slowdown is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotatable armature 230. In an implementation, air transfer device 240 is coupled to the external armature 230 such that rotation of the external armature 230 causes the air transfer device 240 to create an air flow that passes over at least a portion of the motor. In an implementation, the air transfer device 240 includes one or more fan blades coupled to the external armature 230. In an implementation, a plurality of fan blades may be arranged in an annular ring such that the air transfer device acts as an impeller that is rotated by movement of the external rotatable armature. As depicted in FIGS. 1D and 1E, air transfer device 240 may be mounted to an outer surface of the external armature 230, in alignment with the motor 220. The mounting of the air transfer device 240 to the armature 230 allows air flow to be directed toward the main portion of the external rotatable armature 230, providing a cooling effect during use. In an implementation, the air transfer device 240 directs air flow such that a majority of the external rotatable armature 230 is in the air flow path.

Further, referring to FIGS. 1D and 1E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases 327 from canister system 300 are directed toward power supply 180 and toward compression system 200. In an implementation, base 315 of canister system 300 receives the vented gases from the canisters. The vented gases 327 are directed through base 315 toward outlet 325 of the base 315 and toward power supply 180. The vented gases, as noted, are cooled due to decompression of the gases and therefore passively provide cooling to the power supply. When the compression system 200 is operated, the air transfer device 240 will gather the cooled vented gases and direct the gases toward the motor 220 of compression system 200. Fan 172 may also assist in directing the vented gas across compression system 200 and out of the housing 170. In this manner, additional cooling may be obtained without requiring any further power requirements from the f1.

### Canister System

Oxygen concentrator 100 may include one or more, such as at least two canisters, each canister may include a gas separation adsorbent. The canisters of oxygen concentrator 100 may be formed from a molded housing. In an implementation, canister system 300 includes two housing components 310 and 510, as depicted in FIG. 1I. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be molded using injection molding, compression molding, or Thixomolding® technology, or the housing components may be die cast. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent or laser welded together.

As shown, valve seats 322, 324, 332, and 334 and air pathways of conduits 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled together separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plug to seal the passage. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, filter 129 may be placed into respective canister receiving portions of housing components 310 and 510 facing the inlet of the respective canisters. The filter 129 removes particles from the feed gas stream entering the canisters.

In some implementations, pressurized air from the compression system 200 may enter air inlet 306. Air inlet 306 is coupled to inlet conduit 330. Air enters housing component 310 through inlet 306 travels through conduit 330, and then to valve seats 322 and 324. FIG. 1J and FIG. 1K depict an end view of housing 310. FIG. 1J depicts an end view of housing 310 prior to fitting valves to housing 310. FIG. 1K depicts an end view of housing 310 with the valves fitted to the housing 310. Valve seats 322 and 324 are configured to receive inlet valves 122 and 124 respectively. Inlet valve 122 is coupled to canister 302 and inlet valve 124 is coupled to canister 304. Housing 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing 310 into canister 302. Similarly valve seat 324 includes an opening 375 that passes through housing 310 into canister 302. Air from conduit 330 passes through openings 323 or 375 if the respective valves 322 and 324 are open, and enters a canister.

Check valves 142 and 144 (See FIG. 1I) are coupled to canisters 302 and 304, respectively. Check valves 142 and 144 are one way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing 310.

Oxygen enriched air from either canister travels through conduit 342 or 344 and enters conduit 346 formed in housing 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 1B, gas pressure within the accumulator 106 may be measured by a sensor, such as with an accumulator pressure sensor 107. (See also FIG. 1F.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some implementations, the pressure sensor may alternatively measure pressure of the gas outside of the accumulator 106, such as in an output path between the accumulator 106 and a valve (e.g., supply valve 160) that controls the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen. Canister 302 is vented by closing inlet valve 122 and opening outlet valve 132. Outlet valve 132 releases the vented gas from canister 302 into the volume defined by the end of housing 310. Foam material may cover the end of housing 310 to reduce the sound made by release of gases from the canisters. Similarly, canister 304 is vented by closing inlet valve 124 and opening outlet valve 134. Outlet valve 134 releases the vented gas from canister 304 into the volume defined by the end of housing 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

In an exemplary implementation, a portion of the oxygen enriched air may be transferred from canister 302 to canister 304 when canister 304 is being vented of nitrogen. Transfer of oxygen enriched air from canister 302 to canister 304, during venting of canister 304, helps to further purge nitrogen (and other gases) from the canister. Flow of oxygen enriched air between the canisters is controlled using flow restrictors and valves, as depicted in FIG. 1B. Three conduits are formed in housing component 510 for use in transferring oxygen enriched air between canisters. As shown in FIG. 1L, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 1M. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 1M. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen concentration of gas passing through the chamber.

### Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in an oxygen accumulator 106 prior to being provided to a user. In some implementations, a tube may be coupled to the accumulator 106 to provide the oxygen enriched air to the user. Oxygen enriched air may be provided to the user through an airway delivery device (e.g., a patient interface) that transfers the oxygen enriched air to the user's mouth and/or nose. In an implementation, an outlet may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 1F. In an implementation, expansion chamber 162 may include one or more devices configured to estimate an oxygen concentration of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste. If the bolus can be delivered in this manner, there may be a linear relationship between the prescribed continuous flow rate and the therapeutically equivalent bolus volume required in pulsed delivery mode for a user at rest with a given breathing pattern. For example, the total volume of the bolus required to emulate continuous-flow prescriptions may be equal to 11 mL for each LPM of prescribed continuous flow rate, i.e., 11 mL for a prescription of 1 LPM; 22 mL for a prescription of 2 LPM; 33 mL for a prescription of 3 LPM; 44 mL for a prescription of 4 LPM; 55 mL for a prescription of 5 LPM; etc. This amount is generally referred to as the LPM equivalent bolus volume. It should be understood that the LPM equivalent may vary between oxygen concentrators due to differences in construction design, tubing size, chamber size, etc. The LPM equivalent will also vary depending on the user's breathing pattern (e.g. breathing rate).

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. Published Patent Application No. 2009-0065007, published Mar. 12, 2009, and entitled “Oxygen Concentrator Apparatus and Method”, which is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185.

In some implementations, ultrasonic sensor 165 and, for example, flow rate sensor 185 may provide a measurement of an actual amount of oxygen being provided. For example, flow rate sensor 185 may measure a volume of gas (based on flow rate) provided and ultrasonic sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc. prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

Oxygen enriched air may be provided to a user through conduit 192. In an implementation, conduit 192 may be a silicone tube. Conduit 192 may be coupled to a user using an airway delivery device 196, as depicted in FIGS. 1G and 1H. Airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device 196 (e.g., a nasal cannula). As depicted in FIG. 1H, oxygen enriched air may be provided to a user through both a nasal airway delivery device 196 and a mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, a mouthpiece 198 is used in combination with a nasal airway delivery device 196 (e.g., a nasal cannula) to provide oxygen enriched air to a user, as depicted in FIG. 1H. Both mouthpiece 198 and nasal airway delivery device 196 are coupled to an inhalation sensor. In one implementation, mouthpiece 198 and nasal airway delivery device 196 are coupled to the same inhalation sensor. In an alternate implementation, mouthpiece 198 and nasal airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal airway delivery device 196) proximate to which the onset of inhalation was detected. Alternatively, oxygen enriched air may be provided to both mouthpiece 198 and nasal airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 may include one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, such as the Internet. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system such as when the valves are implemented as electro-mechanical valves (e.g., any one or more of valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g. programmed by program instructions) to operate oxygen concentrator 100 and is further configured to monitor the oxygen concentrator 100 for malfunction states. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

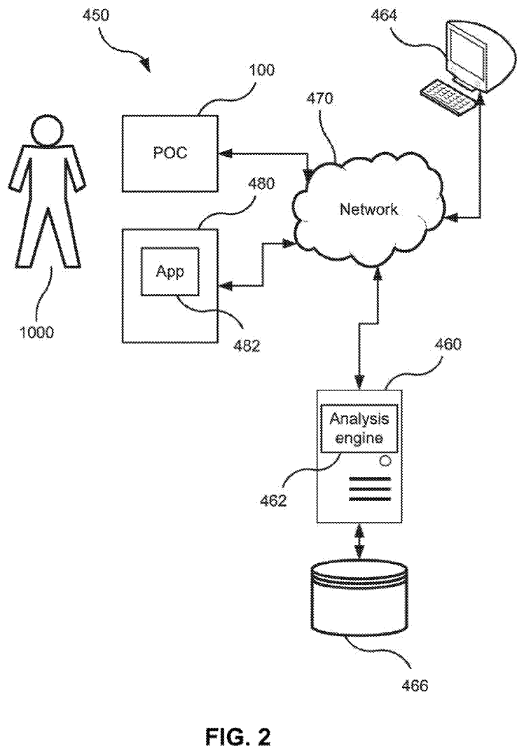

FIG. 2 illustrates one implementation of a connected POC therapy system 450 including the POC 100. Controller 400 of the POC 100 (see FIG. 1B) includes a transceiver 430 configured to allow the controller 400 to communicate, using a wireless communication protocol such as the Global System for Mobile Telephony (GSM) or other protocol (e.g., WiFi), with a remote computing device such as a cloud-based server 460 such as over a network 470. The network 470 may be a wide-area network such as the Internet, or a local-area network such as an Ethernet. The controller 400 may also include a short range wireless module in the transceiver 430 configured to enable the controller 400 to communicate, using a short range wireless communication protocol such as Bluetooth™, with a portable computing device 480 such as a smartphone. The portable computing device, e.g. smartphone, 480 may be associated with a user 1000 of the POC 100.

The server 460 may also be in wireless communication with the portable computing device 480, such as a smartphone, using a wireless communication protocol such as GSM. A processor of the smartphone 480 may execute a program 482 known as an “app” to control the interaction of the smartphone 480 with the user 1000, the POC 100, and/or the server 460. The server 460 may have access to a database 466 that stores operational data about the POC 100 and user 1000.

The server 460 includes an analysis engine 462 that may execute methods of operating and monitoring the POC 100 as further described below. The server 460 may also be in communication via the network 470 with other devices such as a personal computing device workstation 464 via a wired or wireless connection. A processor of the personal computing device 464 may execute a “client” program to control the interaction of the personal computing device 464 with the server 460. One example of a client program is a browser.

In a further implementation, the server 460 may be configured to host a portal system. The portal system may receive, from the portable computing device 480 or directly from the POC 100, data relating to the operation of the POC 100. As described above, the personal computing device 464 may execute a client program such as a browser to allow a user of the personal computing device 464 (such as a representative of an HME) to access the operational data of the POC 100, and other POCs in the connected POC therapy system 450, via the portal system hosted by the server 460. In this fashion, such a portal system may be utilised by an HME to manage a population of users of POC devices, e.g. the POC 100, in the connected POC therapy system 450. The portal system may provide actionable insights into user or device condition for the population of POC devices and their users based on the operational data received by the portal system. Such insights may be based on rules that are applied to the operational data.

In some implementations, the controller 400 of the POC may be configured to implement supply valve control to regulate bolus size (volume) in the system, which may optionally be implemented without use of a flow rate sensor of the POC. For example, the POC may be equipped with a pressure sensor, such as the pressure sensor 107 in the accumulator downstream of the sieve beds, and regulate bolus size, generated by the POC, as a function of pressure. Such regulation of bolus size may be a function of accumulator pressure.

Further functions that may be implemented with or by the controller 400 are described in detail in other sections of this disclosure.

### Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, dosage buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button is activated to turn the system off, controller 400 may initiate a shutdown sequence to place the system in a shutdown state (e.g., a state in which both canisters are pressurized). Dosage buttons 620, 622, 624, and 626 allow the prescribed continuous flow rate of oxygen enriched air to be selected (e.g., 1 LPM by button 620, 2 LPM by button 622, 3 LPM by button 624, and 4 LPM by button 626). Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing button 630 for active mode or button 635 for sleep mode. The adjustments made by the oxygen concentrator 100 in response to active mode or sleep mode being activated are described in more detail herein.

### Controlled Release of Oxygen Enriched Air

The main use of an oxygen concentrator 100 is to provide supplemental oxygen to a user. Generally, the continuous flow rate of supplemental oxygen to be provided is prescribed by a physician. Typical prescribed continuous flow rates of supplemental oxygen may range from about 1 LPM to up to about 10 LPM. The most commonly prescribed continuous flow rates are 1 LPM, 2 LPM, 3 LPM, and 4 LPM.

In order to minimize the amount of oxygen enriched air that is needed to be produced to emulate the prescribed continuous flow rate, controller 400 may be programmed to synchronize release of the oxygen enriched air with the user's inhalations, according to a therapy mode known as pulsed oxygen delivery (POD) or demand oxygen delivery. Releasing a bolus of oxygen enriched air to the user as the user inhales may prevent unnecessary oxygen generation (further reducing power requirements) by not releasing oxygen, for example, when the user is exhaling. Reducing the amount of oxygen required may effectively reduce the amount of air compression needed by oxygen concentrator 100 and consequently may reduce the power demand from the compressors.

Oxygen enriched air produced by oxygen concentrator 100 may be stored in an oxygen accumulator 106 and, in POD mode, released to the user as the user inhales. The amount of oxygen enriched air provided by the oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the amount of oxygen required to emulate the prescribed continuous flow rate of a user, the oxygen enriched air may be provided as a bolus soon after the onset of a user's inhalation is detected. For example, the bolus of oxygen enriched air may be provided in the first few milliseconds of a user's inhalation.

In an implementation, a sensor such as a pressure sensor 194 may be used to determine the onset of inhalation by the user. For example, the user's inhalation may be detected by using pressure sensor 194. In use, conduit 192 for providing oxygen enriched air is coupled to a user's nose and/or mouth through the nasal airway delivery device 196 and/or mouthpiece 198. The pressure in conduit 192 is therefore representative of the user's airway pressure. At the onset of an inhalation, the user begins to draw air into their body through the nose and/or mouth. As the air is drawn in, a negative pressure is generated at the end of the conduit 192, due, in part, to the venturi action of the air being drawn across the end of the conduit. Controller 400 analyses the pressure signal from the pressure sensor 194 to detect a drop in pressure indicating the onset of inhalation. Upon detection of the onset of inhalation, supply valve 160 is opened to release a bolus of oxygen enriched air from the accumulator 106. A positive change or rise in the pressure indicates an exhalation by the user, upon which the release of oxygen enriched air is discontinued. In one implementation, when a positive pressure change is sensed, supply valve 160 is closed until the next onset of inhalation is detected. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

### Additional Exemplary Oxygen Concentrator and Canister System Implementations

In portable oxygen concentrators (“POC”), including implementations such as oxygen concentrator described above in FIG. 1A to FIG. 2, the canister system, also referred to as a sieve bed assembly, have typically been replaced every year. The need to replace the canister system has primarily been due to degradation of the gas separation adsorbent (e.g., zeolite) and/or the desiccant materials, which once degraded, exhibit reduced performance of the sieve bed and lower levels of oxygen separation. Another related degradation issue for sieve bed assemblies can include the fluidization of the sieve bed materials.

The replacement process has conventionally involved replacing the whole canister system or sieve bed assembly, which includes many components, often conducted by a service engineer or less frequently by the end user (e.g., the patient). It is contemplated that the sieve bed itself within the assembly may need to be replaced earlier than expected or to otherwise degrade sooner than the other components of the overall sieve bed assembly. For example, the sieve bed includes both the gas separation adsorbent, such as zeolite, in fluid connection with the outlet end of the sieve bed assembly and a desiccant material for adsorption of water (where the water may be in the form of a liquid, gas or mixture thereof) in fluid connection with the inlet end of the sieve bed assembly. The sieve bed itself operates to separate the gases introduced from the air stream (or feed gas) into the inlet end of the sieve bed assembly via the oxygen concentrator.

The presently described technology contemplates a more modular system for a sieve bed assembly where the desiccant is contained within its own user-replaceable receptacle that can easily be removed from a housing assembly for the sieve bed assembly by the end-user (e.g., including a caregiver or healthcare professional) of an oxygen concentrator and replaced with a new user-replaceable receptacle. As such, the sieve bed assembly comprises a user-replaceable desiccant receptacle (or receptacle). The receptacle may be in the form of a rigid or semi-rigid cartridge, a flexible basket or netting or otherwise permeable fabric, or combinations thereof, that retains the desiccant. For example, the receptacle may include a rigid housing.

The desiccant is the more economical component in the sieve bed assembly in comparison with a gas separation adsorbent, such as a zeolite or other type of molecular sieve. Furthermore, the desiccant is generally considered a sacrificial component in the sieve bed assembly as it is intended to remove water from the air stream (i.e. feed gas) entering through the inlet to minimize the gas separation adsorbent from being exposed to such water. This is especially desirable since it is less economical to replace, for example, a zeolite, than the desiccant. Without the desiccant minimizing the exposure of the gas separation adsorbent to water, water would adsorb onto, for example, the zeolite, and reduce the ability of the gas separation adsorbent to adsorb nitrogen, such as in the case of an oxygen concentrator. The gas separation adsorbent in the context of the oxygen concentrator is operable to selectively adsorb nitrogen within the pressure conditions created in the sieve bed during operation of the oxygen concentrator. Then, as the nitrogen is adsorbed, the remaining gas from the processed air stream becomes oxygen-enriched for delivery to the end-user of the oxygen concentrator, and the separated nitrogen is vented to the atmosphere.

A removable and user-replaceable desiccant receptacle is particularly desirable and efficient because it allows the desiccant to be replaced once it is degraded without having to replace the gas separation adsorbent or other sieve bed assembly components that may have additional operational life. In addition, the removable user-replaceable desiccant receptacle may be removed after the POC is used each time so as to minimize diffusion of water (such as moisture) from the removable desiccant receptacle to the gas separation adsorbent within the canister.

Another desirable aspect of the present technology is that a user-replaceable receptacle can be monitored by the end-user, including a healthcare professional or caregiver. This can be accomplished by a sensor within the user-replaceable receptacle or on the wall of the user-replaceable receptacle, along with a circuit board communicatively connected to the POC controller, such as controller 400. In some implementations, the sensor is connected to a controller (such as controller 400) and/or communications interface configured to transmit an electronic signal to a communications network including information for ordering a replacement user-replaceable cartridge. In some implementations, the sensor may be a moisture sensor that monitors the moisture content of the desiccant to provide indication of the desiccant degradation. In some implementations, the sensor may be an optical sensor that can be used with optic(s) extending from the optical sensor. The optical sensor can be disposed in or on the desiccant receptacle and have a visual line-of-sight to the exterior of the housing of the POC where the optical pathway is within a wall, if any, of the desiccant receptacle. The optical pathway can further extend through the housing of the sieve bed assembly and to the outer housing of the POC. In some implementations, the sensor can be configured via the controller to notify the end-user (e.g., including the caregiver or healthcare professional) or the user-replaceable desiccant receptacle supplier that the useful life of the desiccant receptacle has been reached. Where the supplier is notified, this can in turn result in an order of a new user-replaceable desiccant receptacle being placed and shipped to the end-user.

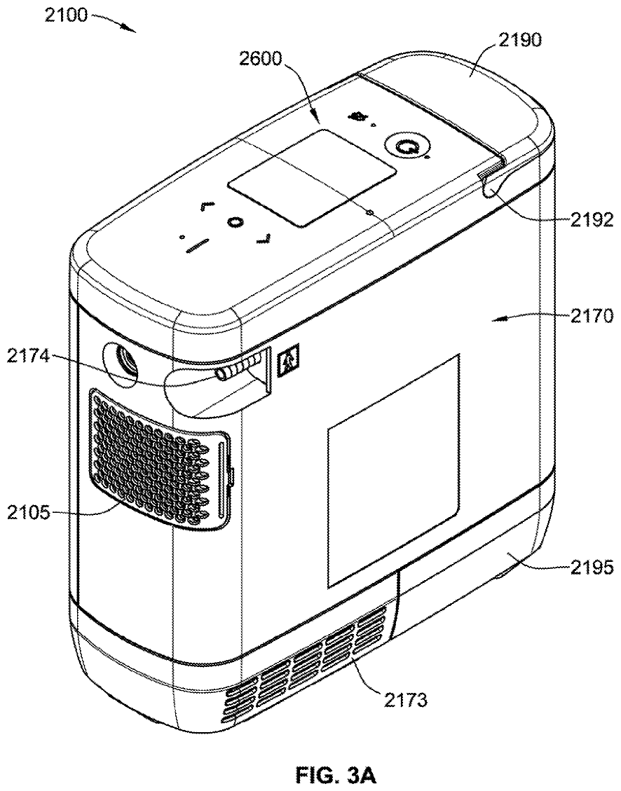

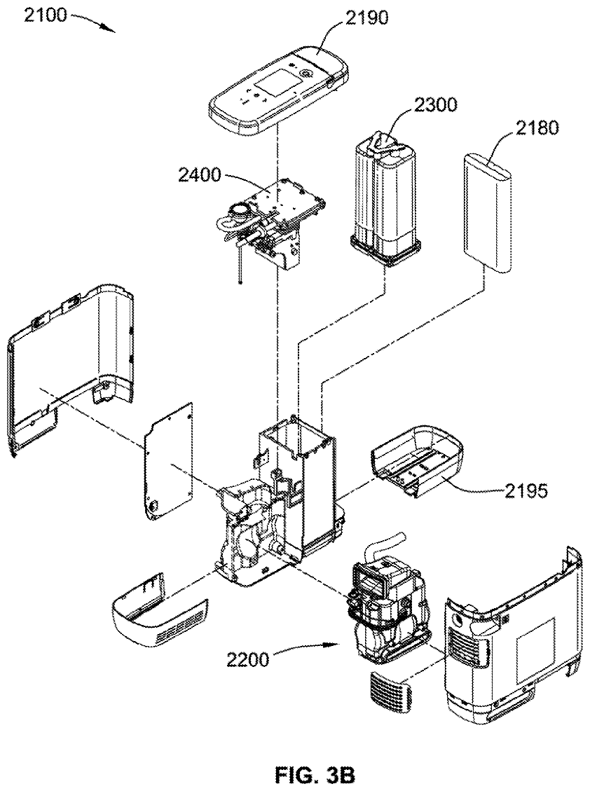

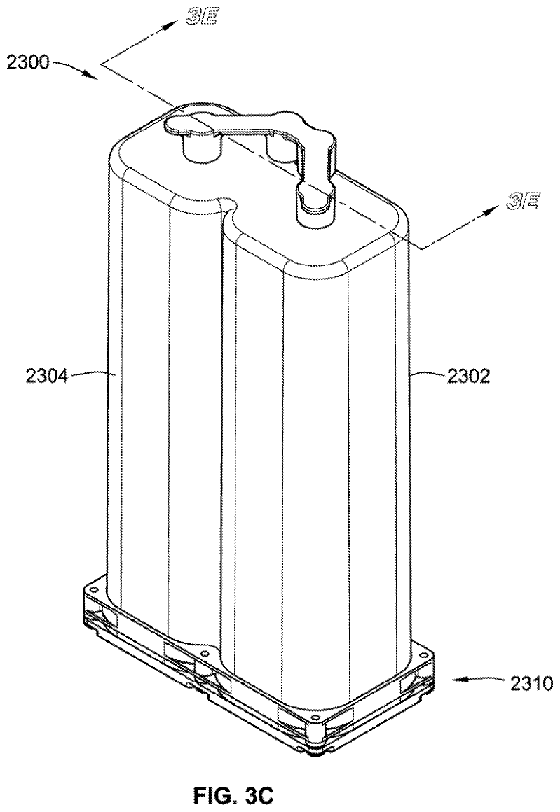

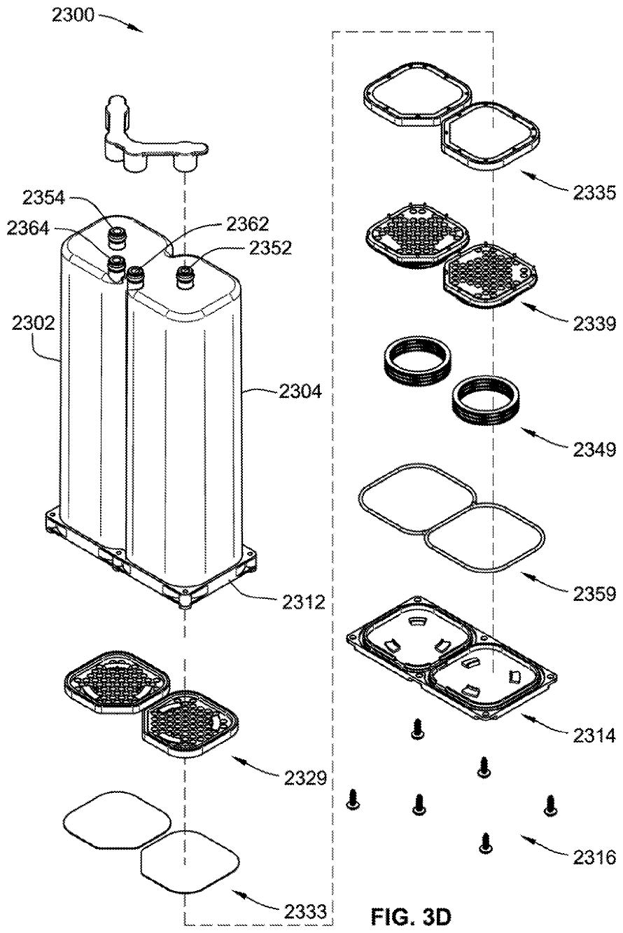

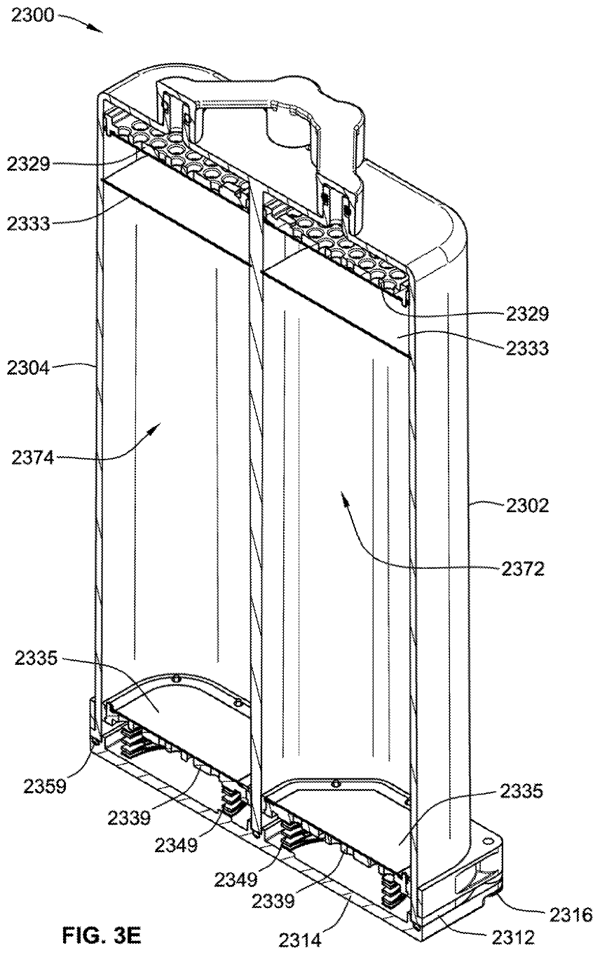

Additional information about the above described technology is discussed below, including in the context of FIGS. 3-15.

Turning now to FIG. 3A, a POC 2100 is depicted including an outer housing 2170 in accordance with another exemplary form of the present technology. In some implementations, outer housing 2170 comprises a light-weight plastic. Outer housing 2170 includes removable panel 2105, cooling system outlets 2173 at select locations of outer housing 2170 (e.g. at a lower half (or bottom end) of the housing 2170), an outlet port 2174, and control panel 2600. Removable panel 2105 comprises two sets of openings (not shown). A first set of openings allow air (e.g., feed gas) to move to the compression system inlets which lead to the compressor, and a second set of openings allow air to enter the housing 2170 to aid in cooling of POC 2100. The first set of openings associated with the compression system inlets can be disposed on an upper portion of the housing 2170, and the second set of openings can be disposed on a lower portion of the housing 2170. Advantageously, the first set of openings allow air to enter and flow through the housing 2170, and the cooling system outlets 2173 allow air to exit the interior of housing 2170 to aid in cooling of the POC 2100. Outlet port 2174 is used to attach a conduit to provide oxygen enriched air produced by the POC 2100 to a user. In some implementations, the outer housing 2170 may include select panels, such as top panel 2190 or bottom panel 2195. The panels may rotate about a hinge, such as hinge 2192 for top panel 2190, or are removable via hidden connectors, to allow access to the interior of the POC 2100, including a sieve bed assembly 2300 and a power supply 2180 (see FIG. 3B). In some implementations, panel 2190 allows access to the power supply 2180 (e.g. a battery) and panel 2195 allows access to the sieve bed assembly 2300. Other removable panels that may be incorporated into a side or end of the POC 2100 are contemplated.

Referring to FIG. 3B, an exploded view is depicted of the main components of the POC 2100 of FIG. 3A. The POC 2100 includes a compression system 2200, a canister assembly (such as sieve bed assembly 2300), an integrated manifold 2400 for connecting to the sieve bed assembly 2300, and a power supply 2180 disposed within the outer housing 2170. Power supply 2180 provides a source of power for the POC 2100. In some implementations and as illustrated in FIG. 3B, sieve bed assembly 2300 and power supply 2180 are in a substantially vertical position relative to the base of the POC 2100.

Other components of the POC 2100 are illustrated and described in FIGS. 3A to 3E that are analogous or similar to some or all of the components described for the POC 100 described in the context of FIGS. 1A to 1N. For example, POC 2100 includes an inlet and a muffler. The muffler may reduce noise of air being drawn in by the compression system 2200 and also may include a desiccant material to initially remove moisture, i.e. water, from the incoming air. The desiccant can further be used to filter the incoming air. POC 2100 may further include a fan used to vent air and other gases from within the POC 2100 to outside the POC 2100 via an outlet, such as the outlet 2173.

Referring to FIG. 3C, a perspective view is depicted of an exemplary sieve bed assembly 2300 for the POC 2100 of FIG. 3A. The sieve bed assembly 2300 may include one or more canisters, such as at least two canisters, wherein each canister may include a gas separation adsorbent. In some implementations, the sieve bed assembly 2300 may comprise a housing that may be die cast or formed using injection molding, compression molding, Thixomolding® technology, or a deep drawing process. The housing may comprise a first housing component (including canister components 2302 and 2304), and a second housing component 2310. The second housing component 2310 can be arranged to cover an open end of the canister components 2302 and 2304. In some implementations, the canister component 2302 may be separate from canister component 2304. Alternatively, and as illustrated in FIG. 3C, the canister component 2302 may be integral with canister component 2304. The second housing component 2310 may be formed separate from the first housing component (e.g., canister components 2302 and 2304) where the first housing component (e.g., canister components 2302, 2304) is coupled to the second housing component 2310 to form the sieve bed assembly 2300.

In some implementations, the sieve bed assembly 2300 may define or more chambers for the sieve bed(s), such as internal chambers 2372 and 2374 (see FIG. 3E). It is contemplated that the second housing component 2310 may be formed as a unitary component such that it may cover the open end of the first housing component (e.g., canister components 2302 and 2304), or may be made up of two parts, wherein each part is adapted to cover the open end of the first housing component (e.g., canister component 2302 or 2304).

In some implementations, housing components 2302, 2304, 2310 may be made from a thermoplastic polymer such as polycarbonate, methylene carbide, polystyrene, acrylonitrile butadiene styrene (ABS), polypropylene, polyethylene, or polyvinyl chloride. As such, the housing components 2302, 2304, 2310 may be molded plastic components. In another implementation, housing components 2302, 2304, 2310 may be made of a thermoset plastic or metal (such as stainless steel or a lightweight aluminum alloy). Lightweight materials may be used to reduce the weight of the POC 2100. In some implementations, the housing components 2302, 2304, 2310 may be fastened together using screws, bolts, or between component 2310 and components 2302, 2304 using easy-release fasteners. Alternatively, one or more of housing components 2302, 2304, and 2310 may be solvent or laser welded together.

Referring to FIG. 3D, an exploded view is depicted of the main components of the exemplary sieve bed assembly 2300 of FIG. 3C. Each of the canister components 2302, 2304 includes an inlet port and an outlet port, wherein the inlet port and the outlet port are arranged on a same end of the canister components 2302, 2304 of the sieve bed assembly 2300. In other words, the outlet of the canister is on the same end as the inlet of the canister. In some implementations, it is contemplated that the inlet ports of canister components 2302, 2304 may be ports 2354, 2352, and the outlet ports of canister components 2302, 2304 may be ports 2364, 2362. In some implementations, the inlet ports of canister components 2302, 2304 may be ports 2364, 2362, and the outlet ports of canister components 2302, 2304 may be ports 2354, 2352. The integrated manifold 2400 (see FIG. 3B) is configured so that it may couple to the inlet ports and outlet ports of the sieve bed assembly 2300. For instance, the integrated manifold 2400 can be disposed adjacent to a top end of the sieve bed assembly 2300 where the ports protrude from canister components 2302, 2304. The integrated manifold 2400 may comprise one or more couplings that permit pneumatic sealing of the inlets ports or the outlet ports of the canister components 2302, 2304. For instance, the one or more couplings may be configured as orifices to receive the inlet ports or the outlet ports of the canister components 2302, 2304.

The exploded view of FIG. 3D depicts additional stacked elements disposed in interior chamber(s), such as internal chambers 2372 and 2374 (See FIG. 3E), defined by the housing components 2302, 2304, 2310. The stacked elements include diffusers 2329, separator layers 2333, gas separation adsorbent base 2335, baffles 2339, and springs 2349. The diffusers 2329 operate similarly to baffles 2339. The diffusers 2329 and the baffles 2339 promote even distribution of the air flowing through the interior chamber(s). In some implementations, the diffusers 2329 and the bafflers 2339 may be able to filter away solid particles that may be present in the air. Housing component 2310 includes perimeter seals 2359, end cover 2314, and screws 2316 that form a compression fitting to secure the open end of unassembled sieve bed assembly 2300 at flange 2312. When the sieve bed assembly 2300 is in a substantially vertical position relative to the base of the POC 2100, the cover 2314 is arranged at a bottom end of the sieve bed assembly 2300. In some implementations, the screws 2316 may be used with washers (not shown), such as spring loaded washers, so that the load of each screw 2316 may be evenly distributed. Each washer may be a generally thin plate with a hole, wherein the hole is adapted to receive each screw 2316. Perimeter seals 2359 assist with creating a seal at the open end of unassembled sieve bed assembly 2300 once the compression fitting is secured between cover 2314 and the flange 2312.

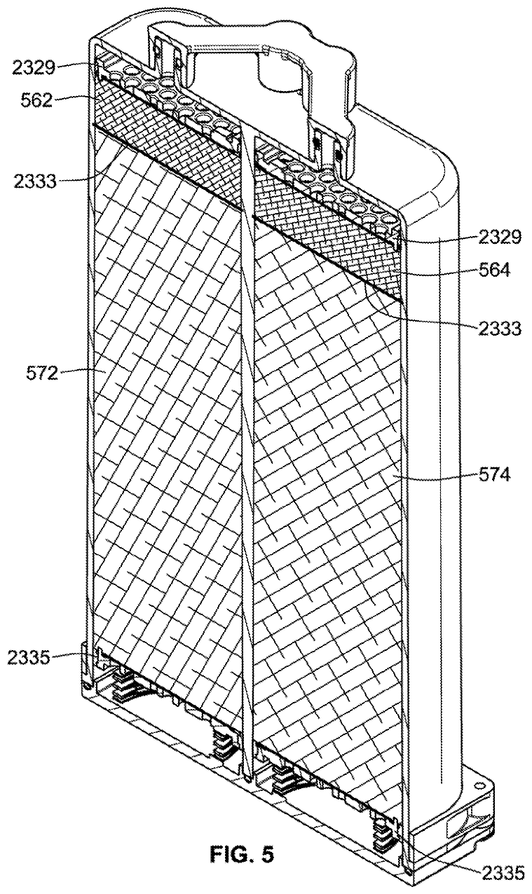

Referring to FIG. 3E, a perspective view is depicted of a cross-section of the sieve bed assembly 2300 of FIG. 3C. The different stacked elements from FIG. 3D are depicted in their assembled positions within an interior chamber, such as internal chambers 2372 and 2374, defined by the housing components 2302, 2304, 2310. The sieve bed assembly 2300 includes two open internal chambers 2372 and 2374 for a gas separation adsorbent (see FIG. 5) to be disposed between gas separation adsorbent base 2335 and the separator layer 2333 in each chamber 2372, 2374. A user-replaceable desiccant receptacle is disposed between the separator layer 2333 and the diffuser 2329 in each chamber 2372, 2374.

The sieve bed assembly 2300 is contemplated to include a number of air pathways that may be integrated into the housing canister components 2302, 2304 to reduce the number of sealed connections needed throughout the air flow of the POC 2100. In some implementations, prior to coupling any housing or sieve bed assembly components together, O-rings may be placed between various components to ensure that the components are properly sealed. In some implementations, some or all of the components, such as housing components 2302, 2304, 2310, may be integrated and/or coupled together separately.

In some implementations, the combination of the baffle 2339 and the spring 2349 may be placed into respective housing components 2302, 2304 with the spring side of the baffle 2339 facing the cover 2314. The combination of the baffle 2339 and spring 2349 may apply force to the gas separation adsorbent in the internal chamber (such as internal chambers 2372 and 2374) while also assisting in minimizing the gas separation adsorbent from entering any exit apertures (such as outlet ports 2362, 2364). Use of a spring 2349 and baffle 2339 may keep the gas separation adsorbent compact while also allowing for expansion (e.g., thermal expansion). Keeping the gas separation adsorbent compact may prevent the gas separation adsorbent from breaking during movement of POC 2100.

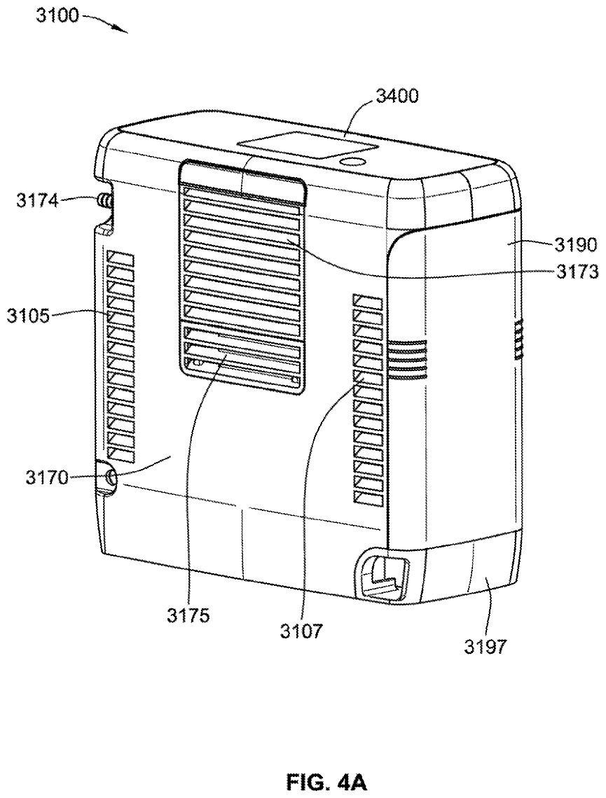

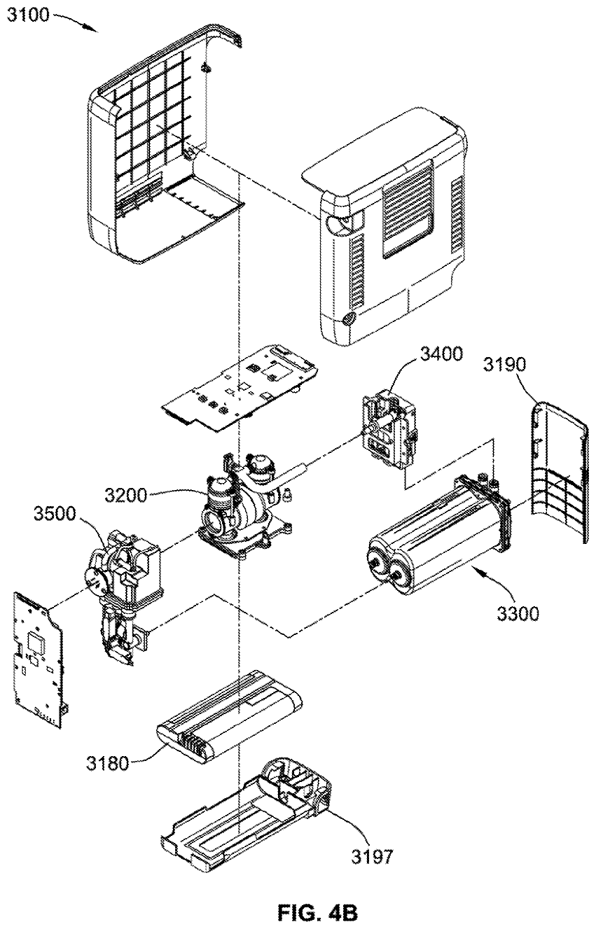

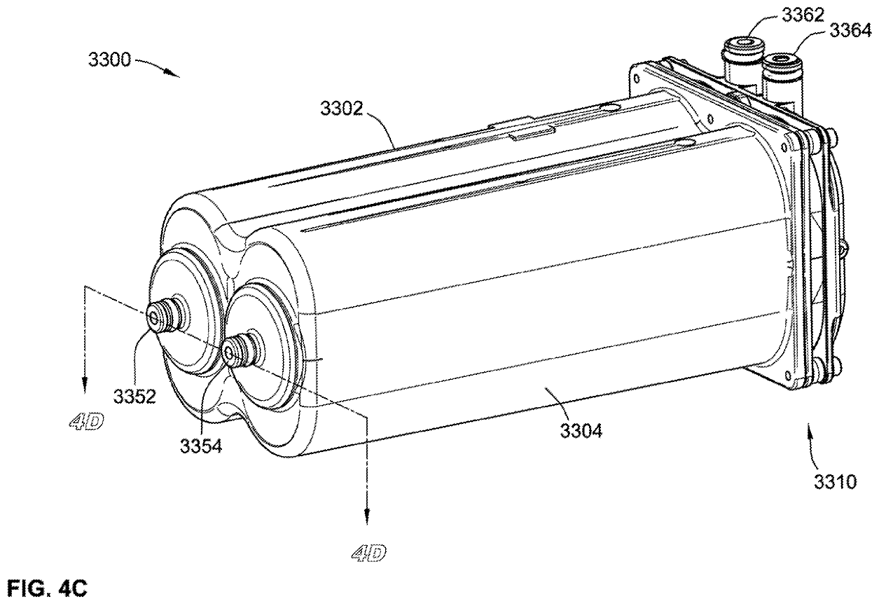

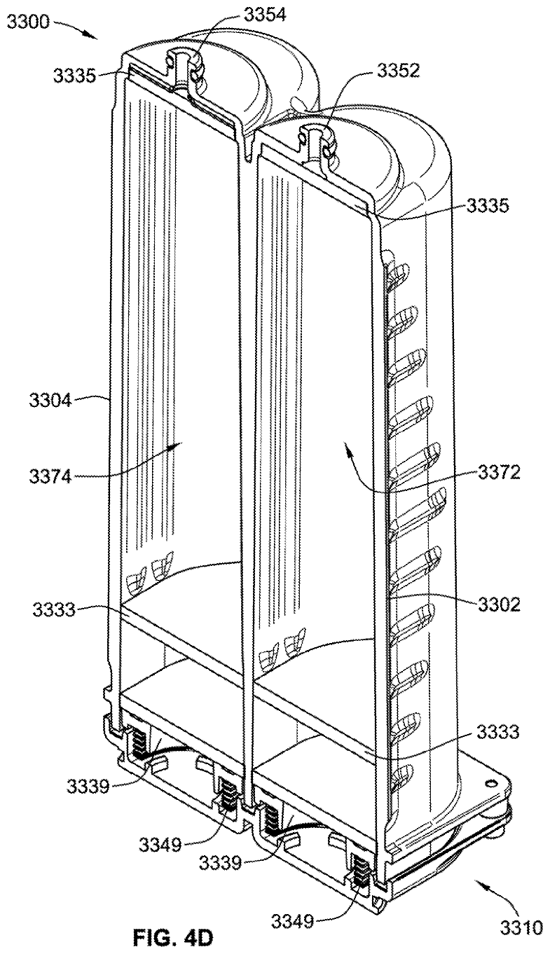

Turning now to FIG. 4A, a POC 3100 is depicted in accordance with yet another exemplary aspect of the present technology. The POC 3100 is depicted including an outer housing 3170. In some implementations, outer housing 3170 may be comprised of a light-weight plastic. Outer housing includes cooling system inlets 3173 and cooling system outlets 3105, 3107 at select locations of outer housing 3170. In addition, the outer housing 3170 can include an outlet port 3174 and a control panel 3400. The outer housing 3170 allows cooling air to enter the POC housing through the cooling system inlets 3173, flow through the compressor system and circulate within the POC, and exit from the interior of housing 3170 through the cooling system outlets 3105, 3107, to aid in cooling of the POC 3100. Outlet port 3174 is used to attach a conduit to provide to a user oxygen enriched air produced by the POC 3100. In some implementation, the outer housing 3170 may include select panels, such as side panel 3190 to allow access to the interior of the POC 3100. In some implementations, a corner panel 3197 also allows access to the interior of the POC, such as a compartment for the power source (e.g., power supply 2180), or panel 3175 may allow access to an air filter for the POC and an opening for feed gas to enter the compressor. Panels in the outer housing 3170 may be hinged where they flip outwardly toward the exterior of the housing or the panels may be removable via hidden connectors. Panels within the outer housing can further allow access to a sieve bed assembly 3300 (see FIG. 4B). Other removable panels are contemplated that may be incorporated into side, end or corner panels of the POC 3100. It is further contemplated that certain faces or sides of the outer housing of POC 3100 may have no openings such that a combinations of openings and no openings in the outer housing provides a targeted air flow in the interior of the POC 3100 that dissipates the heat within the POC 3100, such as heat created by the compression system.

Referring to FIG. 4B, an exploded view is depicted of the main components of the POC 3100 of FIG. 4A. The POC 3100 includes a compression system 3200, a canister assembly (such as sieve bed assembly 3300), an inlet manifold 3400, an outlet manifold 3500, and a power supply 3180 disposed within an outer housing 3170. Power supply 3180 provides a source of power for the POC 3100. In some implementations and as illustrated in FIG. 4B, sieve bed assembly 3300 and power supply 3180 are in a substantially horizontal position relative to the base of the POC 3100.

Other components of the POC 3100 are illustrated and described in FIGS. 4A to 4D that are analogous or similar to some or all of the components described for the POC 100 described in the context of FIGS. 1A-1N and the POC 2100 described in the context of FIGS. 3A-3E. For example, POC 3100 includes an inlet and muffler. The muffler may reduce noise of air being drawn in by the compression system and also may include a desiccant material to initially remove moisture, i.e. water, from the incoming air. POC 3100 may further include a fan used to vent air and other gases from within the POC 3100 to outside the POC 3100 via an outlet.

Referring to FIG. 4C, a perspective view is depicted of an exemplary sieve bed assembly 3300 for the POC 3100 of FIG. 4A. The sieve bed assembly 3300 may include one or more canisters, such as at least two canisters, where each canister may include a gas separation adsorbent, such as a molecular sieve or zeolite. The sieve bed assembly may comprise a housing that may be die cast or formed using injection molding, compression molding, Thixomolding® technology, or a deep drawing process. The housing may be formed by a first housing component, including canister components 3302 and 3304, and a second housing component 3310. The second housing component 3310 is arranged to cover an open end of the first housing component. In some implementations, the canister component 3302 may be distinct or separate from canister component 3304. Alternatively, and as illustrated in FIG. 4C, the canister component 3302 may be integral with canister component 3304. The second housing component 3310 may be formed such that it is separate from the canister components 3302 and 3304 of the first housing component. The second housing component 3310 can be coupled to the first housing component to form the sieve bed assembly 3300. Consequently, the sieve bed assembly 3300 may define one or more chambers for the sieve bed(s), such as internal chambers 3372 and 3374 (see FIG. 4D). In some implementations, the second housing component 3310 may be formed as a unitary component such that it may cover the open end of the first housing component (e.g., canister components 3302 and 3304), or may be made up of two parts, wherein each part is adapted to cover the open end of the first housing component (e.g., canister component 3302 or 3304). The POC 3100 may form a two-part molded plastic frame that defines a housing canister components 3302 and 3304 that are part of sieve bed assembly 3300. Additional housing component 3310 may be formed separately and then coupled together. Housing components 3302, 3304, 3310 may be made from a thermoplastic polymer such as polycarbonate, methylene carbide, polystyrene, acrylonitrile butadiene styrene (ABS), polypropylene, polyethylene, or polyvinyl chloride. As such, the housing components 3302, 3304, 3310 may be molded plastic components. In another implementation, housing components 3302, 3304, 3310 may be made of a thermoset plastic or metal, such as stainless steel or a lightweight aluminum alloy. Lightweight materials may be used to reduce the weight of the POC 3100. In some implementations, the housing components 3302, 3304, 3310 may be fastened together using screws, bolts, or between component 3310 and components 3302, 3304 using easy-release compression fasteners. Alternatively, one or more of housing components 3302, 3304, and 3310 may be solvent or laser welded together.

Referring to FIG. 4C, each of the housing components 3302, 3304 includes an inlet port and an outlet port. The inlet port and the outlet port are arranged at opposite ends of the sieve bed assembly 3300. In other words, the outlet of the canister is at an opposing end to the inlet of the canister. It is contemplated that the inlet ports may be ports 3362, 3364 on the second housing component 3310, respectively, and the outlet ports may be ports 3352, 3354 on housing components 3302, 3304, respectively. In some implementations, the inlet ports may be ports 3352, 3354 on first housing components 3302, 3304, respectively, and the outlet ports may be ports 3362, 3364 on second housing component 3310, respectively. Insertion of the sieve bed assembly 3300 into the POC 3100 may include joining the inlets and/or outlets of the sieve bed assembly 3300 with a coupling of one or more manifolds. A first manifold is configured so that it may couple to the inlets of the sieve bed assembly 3300 and a second manifold is configured so that it may couple to the outlets of the sieve bed assembly 3300. In addition, each manifold may comprise one or more couplings that permit pneumatic sealing of the inlets ports or the outlet ports of the housing components 3302, 3304, 3310. For example, the one or more couplings may be configured as orifices to receive the inlet ports or the outlet ports of the housing components 3302, 3304, 3310.

Referring to one exemplary aspect of the sieve bed assembly 3300 in FIG. 4C in the context of the POC 3100 in FIG. 4B, the ports 3352, 3354 are outlet ports for POC 3100 and ports 3362, 3364 are inlet ports.

Referring to FIG. 4D, an assembled perspective cross-sectional view is depicted of the sieve bed assembly of FIG. 4C. The sieve bed assembly 3300 includes two open internal chambers 3372 and 3374 for a gas separation adsorbent (not shown) to be disposed between gas separation adsorbent base 3335 and the separator layer 3333 in each chamber 3372, 3374. A user-replaceable desiccant receptacle (not shown) can be disposed between the separator layer 3333 and diffuser (not shown) in each chamber 3372, 3374, each of which are in fluid connection with inlet ports 3362, 3364. It is contemplated that the diffuser is operable to evenly distribute and filter air entering the user-replaceable desiccant receptacle. The sieve bed assembly 3300 is contemplated to include a number of air pathways that may be integrated into the housing canister components 3302, 3304 to reduce the number of sealed connections needed throughout the air flow of the POC 3100. In some implementations, prior to coupling any housing or sieve bed assembly components together O-rings may be placed between various components to ensure that the housing components are properly sealed. In some implementations, components may be integrated and/or coupled together separately.

In some implementations, the combination of a baffle 3339 and a spring 3349 may be placed into respective housing components 3302, 3304 with the spring side of the baffle 3339 facing a cover of the second housing component 3310. The combination of the baffle 3339 and the spring 3349 may apply force to the gas separation adsorbent in the internal chamber, such as internal chambers 3372, 3374, while also assisting in minimizing the gas separation adsorbent from entering any exit apertures, such as outlet ports 3352, 3354. Use of a spring 3349 and baffle 3339 may keep the gas separation adsorbent compact while also allowing for expansion (e.g., thermal expansion). Keeping the gas separation adsorbent compact may minimize the gas separation adsorbent from breaking during movement of the POC 3100.

In some implementations, it is contemplated that the internal chambers of the sieve bed assemblies 2300 or 3300 may be pressurized in a swing adsorption process during operation of a portable oxygen concentrator, such as POC 2100 and 3100. It is further contemplated during operation of the POC 2100 and 3100 that the inlets 2352, 3354 are in fluid communication with their respective internal chambers 2374, 3372. Similarly, it is contemplated during operation of the POC 2100 and 3100 that the inlets 2354, 3364 are in fluid communication with their respective internal chambers 2372, 3374.

The inlets and the outlets are arranged at opposite ends of the sieve bed assembly 3300 and at the same end in sieve bed assembly 2300. In some implementations, it is contemplated that the inlet ports may be ports 2362, 3362 in fluid communication with their respective internal chambers 2374, 3372. Similarly, it is contemplated that the inlet ports may be ports 2364, 3364 that are in fluid communication with their respective internal chambers 2372, 3374. In addition, it is contemplated the inlet(s) and outlet(s) for a sieve bed assembly may be disposed anywhere on the housing for the sieve bed assembly.

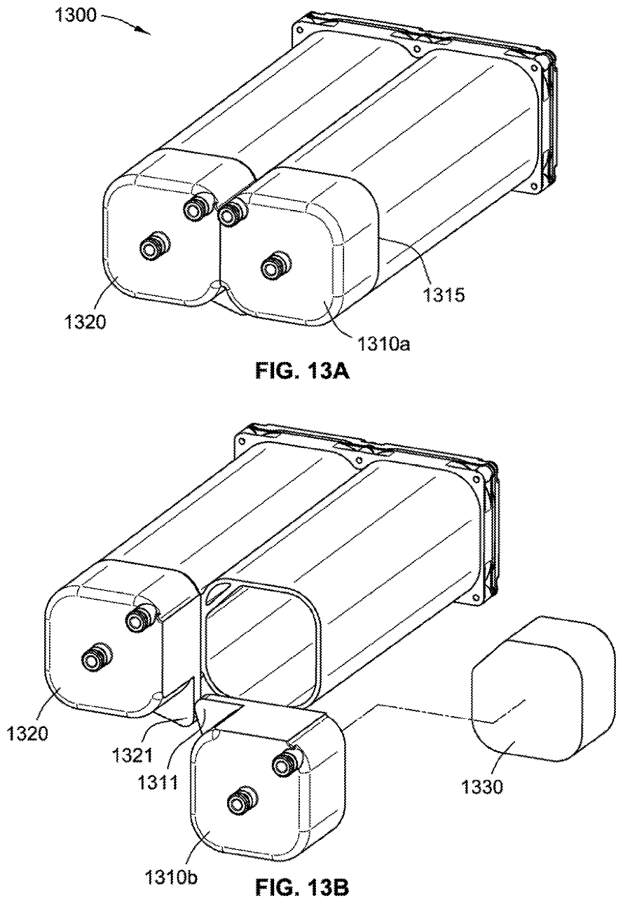

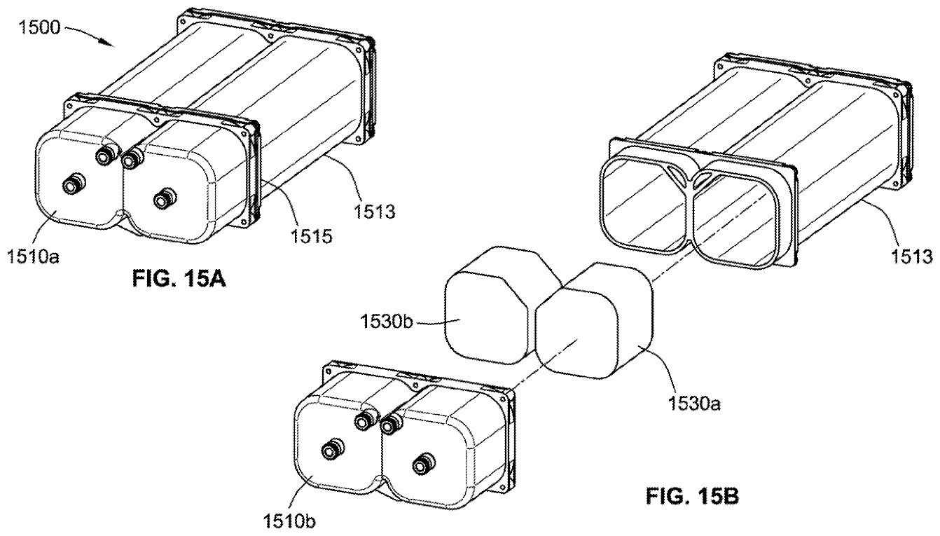

Turning now to FIG. 5, a perspective cross-sectional view is depicted of the exemplary sieve bed assembly 2300 in FIGS. 3C-3E with the addition of user-replaceable desiccant receptacles 562, 564 and gas separation adsorbents 572, 574. A desirable implementation of the present technology is a sieve bed assembly to include a desiccant receptacle that can be replaced by the end-user patient, caregiver, or healthcare professional. It is contemplated that each user-replaceable receptacle 562, 564 is disposed between a diffuser 2329 in fluid connection with the inlet of the sieve bed assembly 2300 and a separator layer 2333 separating the user-replaceable receptacle 562, 564 from the gas separation adsorbent 572, 574. In an implementation, the separator layer 2333 is disposed between the user-replaceable receptacle 562, 564 and the gas separation adsorbent 572, 574. In another implementation, the separator layer 2333 is positioned directly adjacent to the user-replaceable receptacle 562, 564 and the gas separation adsorbent 572, 574. The user-replaceable receptacle (receptacle 562 or 564) and the gas separation adsorbent (adsorbent 572 or 574) are shaped such that they together occupy substantially all of the internal chamber between the diffuser 2329 and the gas separation adsorbent base 2335. Advantageously, the various components, such as the user-replaceable receptacle and the gas separation adsorbent in the internal chamber, are configured such that there may be no or minimal dead space within each canister of the sieve bed assembly 2300. FIGS. 13A, 13B, 15A, and 15B, which are discussed in more detail below, depict two exemplary aspects of how access may be gained to a user-replaceable desiccant receptacle by at least one removable cap, such as rotatable top caps 1310, 1320 and removable access cap 1510, which is incorporated into the sieve bed assembly.

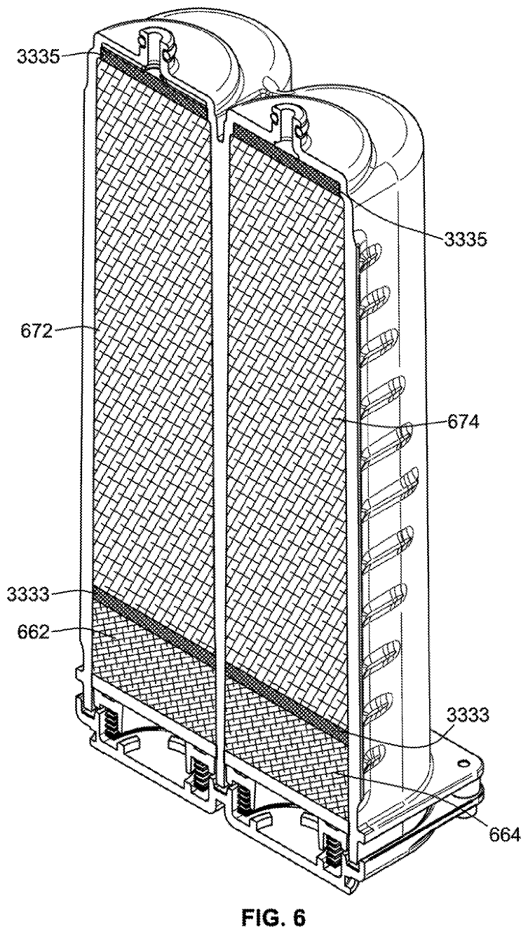

Turning now to FIG. 6, a perspective cross-sectional view is depicted of the exemplary sieve bed assembly 3300 in FIGS. 4C-4D with the addition of user-replaceable desiccant receptacles 662, 664 and gas separation adsorbents 672, 674. A desirable implementation of the present technology is a sieve bed assembly to include a desiccant receptacle that can be replaced by the end-user patient, caregiver, or healthcare professional. It is contemplated that a user-replaceable receptacle (receptacle 662 or 664) is disposed between a diffuser (not shown) in fluid connection with the inlet of the sieve bed assembly and a separator layer 3333 separating the user-replaceable receptacles (receptacle 662 or 664), from the gas separation adsorbent (adsorbent 672 or 674). In an implementation, the separator layer 3333 is disposed between the user-replaceable receptacle 662, 664 and the gas separation adsorbent 672, 674. In another implementation, the separator layer 3333 is positioned directly adjacent to the user-replaceable receptacle 662, 664 and the gas separation adsorbent 672, 674. The user-replaceable receptacle (receptacle 662 or 664), and the gas separation adsorbent (adsorbent 672 or 674), are shaped such that they together occupy substantially all of the internal chamber between the diffuser and the gas separation adsorbent base 3335.

The separator layers 2333, 3333 may include the locking and/or sealing mechanism described below in the context of FIGS. 8-11 to provide a barrier against moisture (i.e. a moisture barrier) between the user-replaceable desiccant receptacle and the gas separation adsorbent. The benefits of minimizing moisture penetration into the gas separation adsorbent include increasing its useful life. The separator layers 2333, 3333 can include different forms such as a housing that contain the gas separation adsorbent or otherwise seals the gas separation adsorbent from air during the replacement of a user-replaceable desiccant receptacle.

It is contemplated that the receptacles 562, 564, 662, 664 may be in the form of a rigid or semi-rigid cartridge (or housing), a flexible basket or netting or otherwise permeable fabric, or combinations thereof, that retain a desiccant material in an interior space of the receptacle. The receptacle may be shaped for, or be capable of taking the shape of, the intended location within the sieve bed assembly 2300, 3300 that is intended for the receptacle. In some implementations, the desiccant may be in a matrix form, a sintered form, a bead form, or combinations thereof. The desiccant can include zeolite, alumina, silica gel, or synthetic crystalline aluminosilicate.

It is contemplated that the gas separation adsorbent 572, 574, 672, or 674 may be in the form of a rigid or semi-rigid cartridge (or housing), or combinations of a rigid and semi-rigid shell, that retains the gas separation adsorbent in an interior space of the cartridge or shell. The gas separation adsorbent may be shaped for, or be capable of taking the shape of, the intended location within the sieve bed assembly 2300, 3300 that is intended for the gas separation adsorbent. In some implementations, the gas separation adsorbent is removable from the housing by an end user of the oxygen concentrator. For example, when the gas separation adsorbent comprises molecular sieve, that may be in a bead form, the gas separation adsorbent can be poured or emptied out of the canister of a sieve bed assembly. Alternatively, the gas separation adsorbent can be contained in a separate receptacle (or cartridge) that is removable from the canister of the sieve bed assembly, such as by at least one removable cap, such as rotatable top caps 1310, 1320 and removable access cap 1510, which is incorporated into the sieve bed assembly. As such, the gas separation adsorbent may be a gas separation adsorbent receptacle or a gas separation adsorbent cartridge.

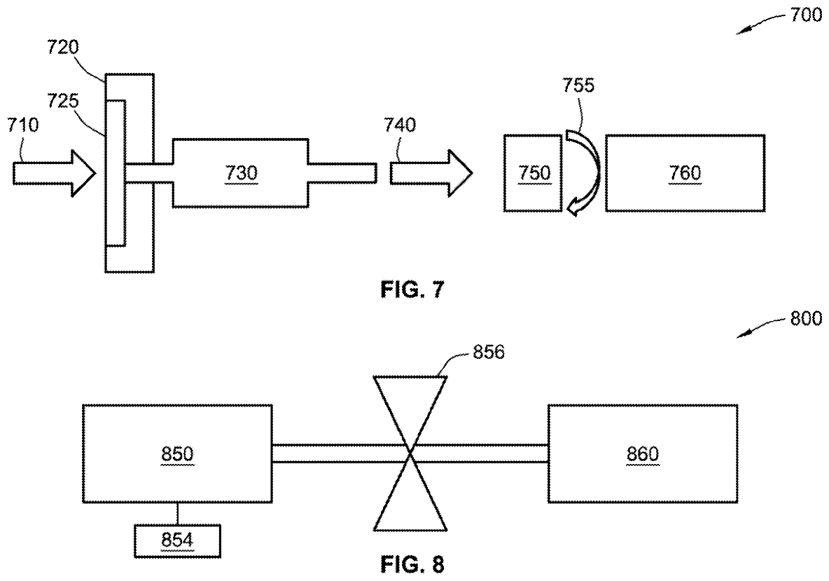

Turning now to FIG. 7, a schematic diagram depicts select exemplary inlet components of a POC 700 for introducing feed gas into an exemplary sieve bed assembly. As discussed above, in some implementations of the present technology, a sieve bed assembly for a POC includes a user-replaceable desiccant cartridge (such as replaceable desiccant cartridge that can be easily replaced by a patient, caregiver, or healthcare professional) or more broadly, a user-replaceable desiccant receptacle 750, positioned between a compressor 720 and a gas separation adsorbent 760. Prior to entering the compressor 720, the air stream 710 may pass through a filter 725, which may be positioned at the inlet of the compressor 720. Consequently, the filter 725 when present may minimize degradation of the components of the POC 700, such as a desiccant, molecular sieve or gas separation adsorbent. The compressor 720 may have a muffler 730 at its outlet end so that the air may pass through the muffler 730 before exiting from the compressor 720 and become air stream 740, which then enters into the user-replaceable desiccant receptacle 750.

It is contemplated that the user-replaceable desiccant receptacle 750 may be coupled to one or more components within the canister or one or more housing components of the sieve bed assembly.

By referring to the receptacle as being user-replaceable or end-user/patient replaceable, it is meant that the receptacle can be easily replaced by a user, patient, caregiver, or healthcare professional, without the need for a service technician or having to replace the entire sieve bed assembly when the desiccant material has become saturated beyond its useful life to effectively protect the gas separation adsorbent.

The sieve bed assembly may comprise a connection mechanism for coupling the user-replaceable receptacle to the canister. The connection mechanism may be integrated with one or more of the canisters, or may be a part of the gas separation adsorbent or may be a part of the user-replaceable desiccant. Connection mechanisms such as a twist, lock and/or seal mechanism are discussed in more detail below. As illustrated in FIG. 7, the user-replaceable desiccant receptacle 750 may be secured to a component within the POC, such as the gas separation adsorbent cartridge 760, by a user-operable connection mechanism comprising a twisting motion, schematically shown as twisting motion 755, for allowing the user to couple (such as, mechanically couple) the user-replaceable desiccant receptacle 750 to the gas separation adsorbent cartridge 760. Consequently, the coupling may move the user-replaceable desiccant receptacle 750 between an unconnected position (or a disconnected position) and a connected position. As will be described in further detail later, the connection mechanism (including the twisting motion 755) may be achieved in various ways.

In some implementations, the connection mechanism may include or be incorporated as part of a separator layer (separator layer 2333, 3333) or be otherwise incorporated into the gas separation adsorbent cartridge 760 and/or user-replaceable desiccant receptacle 750. In some implementations, the connection mechanism may comprise a sealing mechanism, such as a mechanical sealing mechanism. The mechanical sealing mechanism may include a bayonet connector, wherein the bayonet connector can allow a user to mechanically couple the user-replaceable desiccant receptacle 750 to a component (e.g., gas separation adsorbent cartridge) within the housing of the sieve bed assembly by moving the user-operable connection mechanism from a disconnected position to a connected position. As such, the connection mechanism may be configured to seal a second section of an internal chamber of a canister, wherein the second section of the internal chamber may contain a gas separation adsorbent.

In some implementations, the connection mechanism can include a push-in port with an O-ring to seal the mechanical coupling of the user-replaceable receptacle to the component (e.g., gas separation adsorbent) within the housing of the sieve bed assembly.

In some implementations, the sealing mechanism is configured to seal the second section of the internal chamber following removal of the user-replaceable receptacle from the internal chamber. It is contemplated that the sealing mechanism may be positioned between the user-replaceable receptacle and the gas separation adsorbent to reduce or minimize water from entering the gas separation adsorbent during replacement of the user-replaceable receptacle. In some implementations, operation of the sealing mechanism includes a twisting step followed by a locking step, thereby reducing or minimizing the exposure of the gas separation adsorbent to water.

In some implementations, the gas separation adsorbent 760 may include an inlet adjacent to the user-replaceable receptacle. In some implementations, the inlet of the gas separation adsorbent 760 is positioned directly adjacent to the user-replaceable receptacle. The inlet can include desiccant materials therein that are separate and distinct from the desiccant contained in the user-replaceable receptacle.

In some implementations, a user-replaceable receptacle includes an outlet adjacent to the gas separation adsorbent. In some implementations, the outlet of the user-replaceable receptacle is positioned directly adjacent to the gas separation adsorbent. The desiccant outlet can include a hydrophobic material therein.

Turning now to FIGS. 8 to 11, exemplary connection mechanisms (including locking and/or sealing mechanisms) are depicted for coupling a user-replaceable desiccant receptacle and a canister having a gas separation adsorbent cartridge. As mentioned above, in some aspects, the user-replaceable receptacle may connect to the gas separation adsorbent cartridge using a connection mechanism, such as a twist-and-seal mechanism. In some implementations, a twisting motion of the twist-and-seal mechanism makes use of a bayonet connector, wherein the bayonet connector can allow a user to mechanically couple the user-replaceable desiccant receptacle 750 to a component (e.g., gas separation adsorbent cartridge) within the housing of the sieve bed assembly by moving the user-operable connection mechanism from a disconnected position to a connected position. In some implementations, a sealing mechanism of the twist-and-seal mechanism can be in the form of a biased seal. In some implementations, the sealing mechanism may be an electronic sealing mechanism, which may be created using a valve structure disposed between the user-replaceable receptacle and the gas separation adsorbent. In some implementations, the valve structure may be integrated to the housing of both canisters, such that the valve structure extends across both canisters of the sieve bed assembly. In some implementations, the valve structure may be coupled to the user-replaceable desiccant receptacle 750 or the gas separation adsorbent cartridge.

It is contemplated that the sealing mechanism and related mechanical components may be embedded within the separator layer, thereby providing a water-tight (or moisture-tight) enclosure for the gas separation adsorbent when the sealing mechanism is in a sealed position.

In some implementations, the valve structure is an electrical valve. In some implementations, the valve structure is configured to close in response to the connection mechanism being moved from the connected position to the unconnected position. Referring to FIG. 8, an exemplary sealing mechanism is depicted in the form of an electrical valve 856 disposed between the user-replaceable desiccant receptacle 850 and the gas separation adsorbent 860. The valve 856 may be operated by a POC controller, pressure sensor mechanism or other sensing device that detects a disconnection of the user-replaceable receptacle 850 from the gas separation adsorbent or other measurable events just prior to such disconnection (e.g., removal of an outer housing, removal of sieve bed assembly, opening of a cap to the internal chamber of receptacle 850). The valve may be a valve structure disposed between the desiccant receptacle 850 and the gas separation adsorbent, and could be incorporated within the gas separation adsorbent.

In some implementations, a sensor 854 is disposed within the sieve bed assembly, such as within the first housing component, or within the user-replaceable receptacle, or within the gas separation adsorbent. The sensor 854 can be used to monitor water levels (or moisture levels) so that a user can be notified when the desiccant materials are spent and the user-replaceable receptacle needs to be replaced. The sensor could be a moisture sensor that is wired into the POC controller or it could be optical. In some implementations, an optical sensor will have a light pathway to either the exterior of the sieve bed assembly, the outer casing of the POC, or some other location that is convenient for an end-user/patient to check the status of the user-replaceable receptacle 850, order a new desiccant cartridge, replace the desiccant cartridge, or even automatically order a new desiccant cartridge if the POC is connected with a communication system. In some implementations, the sensor is disposed within the housing of the sieve bed assembly to monitor water removal effectiveness of the user-replaceable receptacle.

Alternatively, the sensor 854 may be absent. Accordingly, the user-replaceable receptacle 850 may be replaced when the POC is turned off and/or the valve 856 is in the closed position (or sealed position).

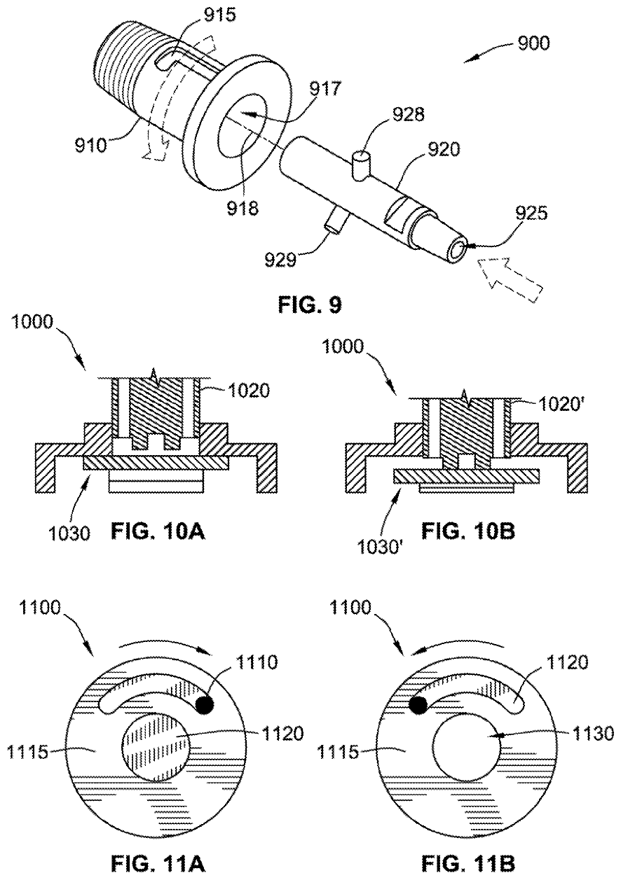

Referring to FIG. 9, an exemplary aspect of the connection mechanism is depicted using a twist and lock mechanism 900. The twist and lock mechanism 900 may comprise a bayonet connector, wherein the bayonet connector is a fastening mechanism consisting of a cylindrical male side with one or more radial pins, and a female receptor with a matching L-shaped slot and a spring to keep the two parts locked together. In some implementations, the cylindrical male side may be a part of the gas separation adsorbent (such as in the form of a gas separation adsorbent cartridge) or the user-replaceable desiccant (such as in the form of a user-replaceable desiccant cartridge). Consequently, the female receptor may be a part of the user-replaceable desiccant cartridge or the gas separation adsorbent cartridge, respectively. The mechanism 900 includes an anchor 910 that may be secured into a shell or housing on the entry end of a gas separation adsorbent cartridge or the exit end of a housing or shell of a user-replaceable desiccant cartridge. The anchor 910 includes an L-shaped slot 915. An elongated locking pin 920 is cylindrical in shape and defines a hollow shaft 925 that allows air to flow through the pin 920. The pin 920 includes one or more protrusions 928, 929 for engaging slot 915 when the locking pin 920 is inserted into an opening 917 along the longitudinal axis 918 of the slotted receiving anchor 910.

As mentioned above, the sealing mechanism may be a biased seal to seal the gas separation adsorbent from the atmosphere following removal of the user-replaceable receptacle from the sieve bed assembly. The sealing mechanism may include a spring-biased plate that allows for an open (e.g., unsealed, unconnected) position and a closed (e.g., sealed, connected) position. When the spring-biased plate is present, the sealing mechanism forms a biased seal.

Referring to FIGS. 10A and 10B, an exemplary aspect of a sealing mechanism 1000 comprising a spring-biased plate 1030 (or spring-biased plate 1030′) at or near the air entry end (or inlet) of a gas separation adsorbent (such as in the form of a gas separation adsorbent cartridge) is depicted. The sealing mechanism 1000 further comprises a protruding member configured to engage the spring-biased plate so that the protruding member causes the spring-biased plate to move between a closed position (as indicated by plate 1030′) and an open position (as indicated by plate 1030). The protruding member may be a part of the user-replaceable desiccant receptacle or a part of the canister. In some implementations, the sealing mechanism 1000 is in the closed position when a user-replaceable desiccant receptacle is present. In some implementations, the sealing mechanism 1000 is in the open position when a user-replaceable desiccant receptacle is absent (or removed, such as during replacement of the user-replaceable desiccant receptacle). In some implementations, the protruding member may be a pin 1020 (or pin 1020′), that controls the movement of air flowing through the sealing mechanism 1000. The pin 1020 may be configured to be in an open position such as in FIG. 10A. In the open position, the pin 1020 does not engage or push against the plate 1030. As such, the air flow pathway through the sealing mechanism 1000 is closed, thereby preventing air from entering the gas separation adsorbent. Alternatively, the pin 1020′ may be configured to be in a closed position such as in FIG. 10B. In the closed position, the pin 1020′ engages the plate 1030′ and pushes against the plate 1030′. Such a movement opens the airflow pathway through the sealing mechanism 1000, thereby allowing air to enter the gas separation adsorbent. The movement of the plate from the open position (i.e. plate 1030) to the closed position (i.e. plate 1030′), and vice versa, can be the result of a twisting motion or a push-pull motion being exerted on the pin (pin 1020 refers to the pin in the open position and pin 1020′ refers to the pin in the closed position).

Referring to FIGS. 11A and 11B, an exemplary aspect of another sealing mechanism at or near the air entry end of a gas separation adsorbent (such as in the form of a gas separation adsorbent cartridge) is depicted. The sealing mechanism may comprise a lock assembly 1100, wherein the lock assembly 1100 comprises a first plate 1115 coupled to a second plate 1120. The first plate 1115 may comprise a recess for receiving a latch 1110, wherein the recess is configured to receive the latch 1110, and wherein the latch 1110 is configured to extend from the second plate 1120 to the first plate 1115. The first plate 1115 may further comprise a hole 1130, wherein the hole 1130 is located at a central part of the first plate 1115. The latch 1110 allows the first plate 1115 to be pivotally connected to the second plate 1120 so that the lock assembly 1100 can be moved between a closed position, wherein the hole 1130 is covered by the second plate 1120 (FIG. 11A), and an open position, wherein the hole 1130 is not covered by the second plate 1120 (FIG. 11B). As such, latch 1110 can be rotated via a twisting motion, thereby coupling and decoupling the user-replaceable desiccant receptacle from the gas separation adsorbent cartridge. For example and as illustrated in FIG. 11A, when the latch 1110 is twisted clockwise, the plate 1120 seals (or covers) the hole 1130, thereby causing the decoupling of the user-replaceable desiccant receptacle and the gas separation adsorbent cartridge. Conversely and as illustrated in FIG. 11B, when the latch 1110 is twisted counterclockwise, the plate 1120 exposes the hole 1130, thereby causing the coupling of the user-replaceable desiccant receptacle to the gas separation adsorbent cartridge.

In some implementations, the connection mechanisms described above may be desirable at both the inlet end of a user-replaceable desiccant receptacle before the air stream enters the sieve bed assembly, and at an outlet end of the user-replaceable desiccant receptacle. Such a configuration can be implemented using a combination of sealing mechanisms, such as a twist-and-lock mechanism and a mechanical sealing (or pneumatic) mechanism.

Additional desirable aspects of the present technology include implementation for accessing the user-replaceable receptacle. Some of those features are discussed above in the context of FIGS. 1 to 3 for gaining access to the interior space of the POC 100, 2100, 3100 so the end-user can further access the sieve bed assembly.

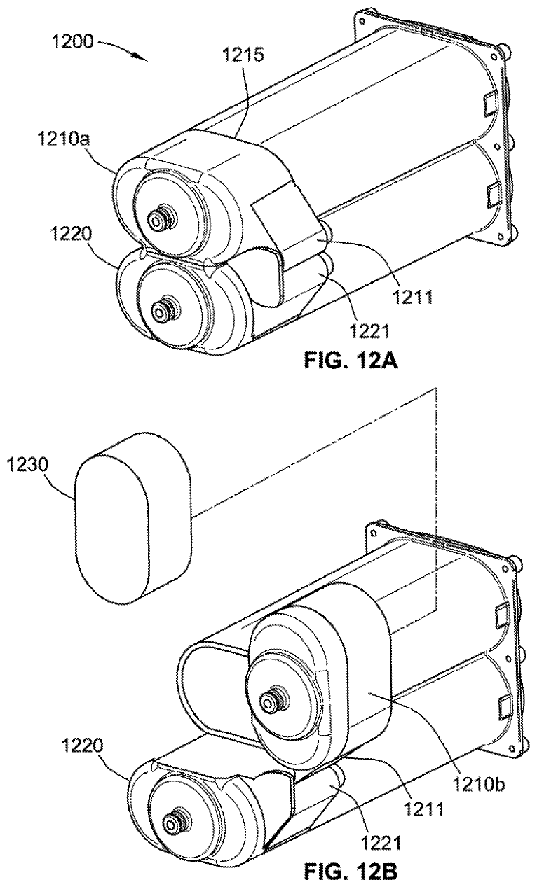

Once the sieve bed assembly is accessed, the user-replaceable desiccant receptacle can be monitored or replaced via an access point of the sieve bed assembly. In some implementations, the second housing component can be removed via a quick-release latch which allows the desiccant receptacle and/or gas separation adsorbent cartridge to slide out of an internal chamber of a canister component of the sieve bed assembly. In some implementations, a removable component such as a door or cap is formed as part of the sieve bed assembly to provide more direct access to the user-replaceable desiccant receptacle without removal of the gas separation adsorbent. If desired, the gas separation adsorbent may also be removed via the removable component. FIGS. 12A to 15B, which are discussed in more detail below, illustrate how access may be gained to the user-replaceable desiccant receptacle by at least one removable component, such as access door 1210, rotatable caps 1310, 1410 and access cap 1510, which is incorporated into the sieve bed assembly.

Turning now to FIGS. 12A and 12B, an exemplary access door 1210 (i.e. access door 1210a which refers to access door 1210 in a closed position, and access door 1210b which refers to access door 1210 in an open position) is depicted for a sieve bed assembly 1200 to allow an end-user to replace a desiccant receptacle 1230. Access doors 1210, 1220 are secured to the canister components of the sieve bed assembly 1200 at hinges 1211, 1221. The access doors 1210, 1220 are configured to swing open about hinges 1211, 1221, as illustrated by access door 1210a in a closed position and then being moved to an open position as depicted by access door 1210b. The desiccant receptacle 1230 may be secured to the access door 1210 where it can readily be removed upon the access door 1210 being swung open. Alternatively, the desiccant receptacle 1230 may remain static on top of (or next to) a gas separation adsorbent that is disposed within an internal chamber of the sieve bed assembly 1200 ready for the end-user to lift it out or decouple the desiccant receptacle 1230 from the gas separation adsorbent. It is contemplated that seals 1215 may be placed along the access door edges to maintain an airtight environment within the interior of the sieve bed assembly 1200 during POC operation.

Turning now to FIGS. 13A and 13B, an exemplary sieve bed assembly 1300 with rotatable top caps 1310 (i.e. rotatable top cap 1310a which refers to rotatable top cap 1310 in a closed position, and rotatable top cap 1310b which refers to rotatable top cap 1310 in an open position), 1320 is depicted for allowing a user (e.g., patient, caregiver, healthcare professional) to gain access to the internal chamber to replace a desiccant receptacle 1330. Rotatable tops caps 1310, 1320 are secured to the housing of the sieve bed assembly 1300 at hinges 1311, 1321. The rotatable top caps 1310, 1320 are configured to rotate about hinges 1311, 1321 as illustrated by cap 1310a in a closed position and then being moved to an open position (i.e. cap 1310b). The desiccant receptacle 1330 is disposed within the rotatable top cap 1310 (as depicted in FIG. 13A as top cap 1310a) where it can readily be removed upon the top cap 1310 being swung open (as depicted in FIG. 13B as top cap 1310b). It is contemplated that seals 1315 are placed along the rotatable top cap edges to maintain an airtight environment within the interior of the sieve bed assembly 1300 during POC operation.

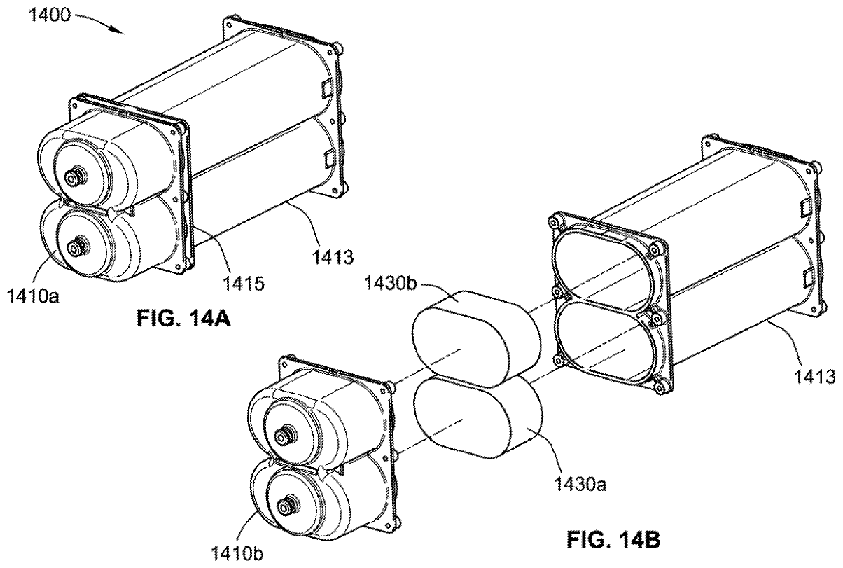

Turning now to FIGS. 14A and 14B, an exemplary sieve bed assembly 1400 with a removable cap 1410 (i.e. cap 1410a which refers to cap 1410 in a closed position, and cap 1410b which refers to cap 1410 in an open position) is depicted for allowing an end-user to gain access to the internal chamber to replace a desiccant receptacle 1430 (desiccant receptacle 1430a or 1430b). The removable cap 1410 illustrated in FIGS. 14A and 14B is configured to contain the inlet ports of the sieve bed assembly 1400. It is contemplated that the removable cap may be positioned at the other end of the sieve bed assembly when the inlet ports and respective desiccant receptacles are located at the other end of the sieve bed assembly (such as the configuration illustrated in FIG. 6). In some implementations and as illustrated in FIGS. 14A and 14B, the removable cap 1410 is shaped to cover both the canisters of the sieve bed assembly 1400. Alternatively, each canister may have its own removable cap, wherein the removable cap is shaped to cover an open end of the canister. In some implementations and as illustrated in FIGS. 14A and 14B, removable cap 1410 is secured at an interface 1415, wherein the interface 1415 is positioned between a main housing component 1413 and a minor housing component of the sieve bed assembly 1400. The removable cap 1410 is configured to be secured and unsecured from the main housing component 1413 of the sieve bed assembly 1400. The end-user replaceable desiccant receptacles 1430a-b are disposed within the removable cap 1410 where they can readily be removed upon the removable cap being decoupled from the main housing portion 1413 of the sieve bed assembly 1400. Alternatively, the desiccant receptacles 1430a-b remain static on top of (or next to) a gas separation adsorbent that is disposed within an internal chamber of the main housing portion 1413 of the sieve bed assembly 1400 and ready for the end-user to lift them out or decouple the desiccant receptacles 1430a-b from the gas separation adsorbent. It is contemplated that the interface 1415 maintains an airtight environment within the interior of the sieve bed assembly 1400 during POC operation when the removable cap 1410 is secured to the main housing portion 1413 of the sieve bed assembly 1400.

Turning now to FIGS. 15A and 15B, another exemplary sieve bed assembly 1500 with removable cap 1510 (i.e. cap 1510a which refers to cap 1510 in a closed position, and cap 1510b which refers to cap 1510 in an open position) is depicted for allowing an end-user to gain access to the internal chamber to replace a desiccant receptacle 1530 (desiccant receptacle 1530a or 1530b). The removable cap 1510 illustrated in FIGS. 15A and 15B is configured to contain the inlet ports and the outlet ports of the sieve bed assembly 1500. In some implementations and as illustrated in FIGS. 15A and 15B, the removable cap 1510 is shaped to cover both the canisters of the sieve bed assembly 1500. Alternatively, each canister may have its own removable cap, wherein the removable cap is shaped to cover an open end of the canister. Removable cap 1510 is a component of the housing of the sieve bed assembly 1500 and is secured at an interface 1515, wherein the interface 1515 is positioned between a main housing component 1513 and a minor housing component of the sieve bed assembly 1500. The removable cap 1510 is configured to be secured and unsecured from the main housing portion 1513 of the sieve bed assembly 1500. The end-user replaceable desiccant receptacles 1530a-b are disposed within the removable cap 1510 where they can readily be removed upon the removable cap 1510 being decoupled from the main housing portion 1513 of the sieve bed assembly 1500. Alternatively, the desiccant receptacles 1530a-b remain static on top of (or next to) a gas separation adsorbent that is disposed within an internal chamber of the main housing portion 1513 of the sieve bed assembly 1500 and ready for the end-user to lift them out or decouple the desiccant receptacles 1530a-b from the gas separation adsorbent. It is contemplated that the interface 1515 maintains an airtight environment within the interior of the sieve bed assembly 1500 during POC operation when the removable cap 1510 is secured to the main housing portion 1513 of the sieve bed assembly 1500.

### Glossary

For the purposes of the present technology disclosure, in certain forms of the present technology, one or more of the following definitions may apply. In other forms of the present technology, alternative definitions may apply.

### General

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%).

Medical Oxygen: Medical oxygen is defined as oxygen enriched air with an oxygen concentration of 80% or greater.

Ambient: In certain forms of the present technology, the term ambient will be taken to mean (i) external of the treatment system or patient, and (ii) immediately surrounding the treatment system or patient.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m²=1 millibar˜0.001 atm). In this specification, unless otherwise stated, pressure is given in units of cmH₂O.

### General Remarks

The term “coupled” as used herein means either a direct connection or an indirect connection (e.g., one or more intervening connections) between one or more objects or components, such as by a pneumatic path. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

Moreover, in interpreting the disclosure, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

In some instances, the terminology and symbols may imply specific details that are not required to practice the technology. For example, although the terms “first” and “second” may be used, unless otherwise specified or indicated by context, they are not intended to indicate any order but may be utilised to merely distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

One or more elements or aspects or steps, or any portion(s) thereof, from one or more of any of claims 1 to 56 below can be combined with one or more elements or aspects or steps, or any portion(s) thereof, from one or more of any of the other claims 1-56 or combinations thereof, to form one or more additional implementations and/or claims of the present disclosure.

1. A sieve bed assembly for a portable oxygen concentrator, the sieve bed assembly including at least one canister, each canister of the sieve bed assembly comprising:
an inlet;
an outlet; and
a housing defining an internal chamber between the inlet and the outlet, the internal chamber comprising a first section disposed adjacent to the inlet, the first section including a user-replaceable receptacle containing a desiccant, the internal chamber further comprising a second section disposed adjacent to the outlet, the second section including a gas separation adsorbent,
wherein the inlet and the outlet are in fluid communication with the internal chamber, and
wherein the user-replaceable receptacle is disposed between the inlet and the gas separation adsorbent to remove water from fluid entering the internal chamber via the inlet.

2. The sieve bed assembly of claim 1, further comprising a connection mechanism for coupling the user-replaceable receptacle to the canister, wherein the connection mechanism is operable between an unconnected position and a connected position.

3. The sieve bed assembly of claim 2, wherein the connection mechanism comprises a sealing mechanism, wherein the sealing mechanism is configured to seal the second section of the internal chamber following removal of the user-replaceable receptacle from the internal chamber.

4. The sieve bed assembly of claim 3, wherein the sealing mechanism is a mechanical sealing mechanism, and wherein the mechanical sealing mechanism includes (i) a bayonet connect or (ii) a spring-biased plate.

5-6. (canceled)

7. The sieve bed assembly of claim 4, wherein the sealing mechanism further includes a protruding member configured to engage the spring-biased plate, wherein the protruding member is configured to move the spring-biased plate between a closed position and an open position.

8. The sieve bed assembly of claim 2, wherein the connection mechanism includes (i) a push-in port with an O-ring to seal the coupling of the user-replaceable receptacle to the canister, or (ii) a twist-lock mechanism for reducing exposure of the gas separation adsorbent to water.

9. (canceled)

10. The sieve bed assembly of claim 2, wherein the sealing mechanism is an electronic sealing mechanism.

11. The sieve bed assembly of claim 10, wherein the electronic sealing mechanism includes a valve structure disposed between the user-replaceable receptacle and the gas separation adsorbent.

12. The sieve bed assembly of claim 11, wherein the valve structure is an electrical valve.

13. The sieve bed assembly of claim 12, wherein the valve structure is configured to close in response to (i) the sealing mechanism being moved from the connected position to the unconnected position, or (ii) an indication that the user-replaceable receptacle is being replaced with another user-replaceable receptacle containing a desiccant.

14. (canceled)

15. The sieve bed assembly of claim 1, wherein the desiccant is in (i) as matrix form or (ii) a sintered form.

16-17. (canceled)

18. The sieve bed assembly claim 1, wherein the gas separation adsorbent is removable from the canister.

19. The sieve bed assembly of claim 1, wherein the gas separation adsorbent is in fluid communication with a gas separation adsorbent inlet disposed between the user-replaceable receptacle and the gas separation adsorbent, the gas separation adsorbent inlet including another desiccant separate and distinct from the desiccant contained in the user-replaceable receptacle.

20. The sieve bed assembly of claim 1, wherein the user-replaceable receptacle includes a desiccant outlet adjacent to the gas separation adsorbent, the desiccant outlet including a hydrophobic material therein.

21. The sieve bed assembly of claim 1, wherein the housing comprises a removable cap to allow a user to gain access to the internal chamber.

22-23. (canceled)

24. The sieve bed assembly of claim 1, further comprising a separator layer disposed between the user-replaceable receptacle and the gas separation adsorbent.

25. The sieve bed assembly of claim 1, further comprising a sensor disposed within the canister to monitor water removal effectiveness of the user-replaceable receptacle.

26. (canceled)

27. The sieve bed assembly of claim 1, wherein the outlet of the canister (i) is on the same end as the inlet of the canister or (ii) at an opposing end to the inlet of the canister.

28. (canceled)

29. A portable oxygen concentrator, comprising:
a compression system including a compressor, wherein the compressor is coupled to a sieve bed assembly, the sieve bed assembly including at least one canister, each canister of the sieve bed assembly comprising:
an inlet;
an outlet; and
a housing defining an internal chamber between the inlet and the outlet, the internal chamber comprising a first section disposed adjacent to the inlet, the first section including a user-replaceable receptacle containing a desiccant, the internal chamber further comprising a second section disposed adjacent to the outlet, the second section including a gas separation adsorbent;
wherein the inlet and the outlet are in fluid communication with the internal chamber;
wherein the user-replaceable receptacle is disposed between the inlet and the gas separation adsorbent to remove water from fluid entering the internal chamber via the inlet.

30. The portable oxygen concentrator of claim 29, further comprising a connection mechanism for coupling the user-replaceable receptacle to the canister, wherein the connection mechanism is operable between an unconnected position and a connected position.

31. The portable oxygen concentrator of claim 30, wherein the connection mechanism comprises a sealing mechanism, wherein the sealing mechanism is configured to seal the second section of the internal chamber following removal of the user-replaceable receptacle from the internal chamber.

32. The portable oxygen concentrator of claim 31, wherein the sealing mechanism is a mechanical sealing mechanism, and wherein the mechanical sealing mechanism includes (i) a bayonet connect or (ii) a spring-biased plate.

33-35. (canceled)

36. The portable oxygen concentrator of claim 30, wherein the connection mechanism includes (i) a push-in port with an O-ring to seal the coupling of the user-replaceable receptacle to the canister, or (ii) a twist-lock mechanism for reducing exposure of the gas separation adsorbent to water.

37-46. (canceled)

47. The portable oxygen concentrator of claim 29, wherein the gas separation adsorbent is in fluid communication with a gas separation adsorbent inlet disposed between the user-replaceable receptacle and the gas separation adsorbent, the gas separation adsorbent inlet including another desiccant separate and distinct from the desiccant contained in the user-replaceable receptacle.

48-51. (canceled)

52. A user-replaceable receptacle for a portable oxygen concentrator comprising:
a containment structure comprising an inlet and an outlet;
a desiccant disposed within the containment structure;
a connection mechanism for coupling the outlet to a gas separation adsorbent, the connection mechanism being operable between an unconnected position and a connected position such that when the connection mechanism is in the connected position, water entering the gas separation adsorbent is reduced.

53-56. (canceled)
